SEQUENCE LISTING (29 sequences)

SEQ ID NO: 1 - protein, 4 aa, synthetic construct
PPTP

SEQ ID NO: 2 - protein, 10 aa, synthetic construct
PEITVTPPTP

SEQ ID NO: 3 - protein, 10 aa, synthetic construct
PVIVITPPTP

SEQ ID NO: 4 - DNA, 22 bp, synthetic construct
  source 1..22: mol_type = other DNA
acagctacaactggagcattta

SEQ ID NO: 5 - DNA, 20 bp, synthetic construct
  source 1..20: mol_type = other DNA
tcagttctgtggccttcttg

SEQ ID NO: 6 - DNA, 18 bp, synthetic construct
  source 1..18: mol_type = other DNA
gctggacttcaacaacct

SEQ ID NO: 7 - DNA, 19 bp, synthetic construct
  source 1..19: mol_type = other DNA
cagacatggcaggagattt

SEQ ID NO: 8 - DNA, 20 bp, synthetic construct
  source 1..20: mol_type = other DNA
gtacgtgtatgacctggaac

SEQ ID NO: 9 - DNA, 21 bp, synthetic construct
  source 1..21: mol_type = other DNA
gagatgtgtctcatggagaag

SEQ ID NO: 10 - DNA, 20 bp, synthetic construct
  source 1..20: mol_type = other DNA
gactgctgtcttggatacac

SEQ ID NO: 11 - DNA, 18 bp, synthetic construct
  source 1..18: mol_type = other DNA
tactgaggagacgcacaa

SEQ ID NO: 12 - DNA, 21 bp, synthetic construct
  source 1..21: mol_type = other DNA
gaaccatctcactgtgtgtaa

SEQ ID NO: 13 - DNA, 21 bp, synthetic construct
  source 1..21: mol_type = other DNA
tggaaaggtttggagtatgtc

SEQ ID NO: 14 - DNA, 20 bp, synthetic construct
  source 1..20: mol_type = other DNA
ccgttcacgtcagtttctac

SEQ ID NO: 15 - DNA, 20 bp, synthetic construct
  source 1..20: mol_type = other DNA
gttggagcaggctcataatc

SEQ ID NO: 16 - DNA, 20 bp, synthetic construct
  source 1..20: mol_type = other DNA
gcactgtaactctggtcttc

SEQ ID NO: 17 - DNA, 19 bp, synthetic construct
  source 1..19: mol_type = other DNA
gggtttggaagaggatcac

SEQ ID NO: 18 - DNA, 21 bp, synthetic construct
  source 1..21: mol_type = other DNA
gatggcatctacatggtacac

SEQ ID NO: 19 - DNA, 19 bp, synthetic construct
  source 1..19: mol_type = other DNA
ggtacaaccttggtggaag

SEQ ID NO: 20 - DNA, 19 bp, synthetic construct
  source 1..19: mol_type = other DNA
cctcatctccatctccctt

SEQ ID NO: 21 - DNA, 21 bp, synthetic construct
  source 1..21: mol_type = other DNA
gttgactaccacgatgatgaa

SEQ ID NO: 22 - DNA, 20 bp, synthetic construct
  source 1..20: mol_type = other DNA
ggagaccgagcagtatatga

SEQ ID NO: 23 - DNA, 20 bp, synthetic construct
  source 1..20: mol_type = other DNA
ctggacattgattgggagag

SEQ ID NO: 24 - DNA, 18 bp, synthetic construct
  source 1..18: mol_type = other DNA
acggcgagttcatctaca

SEQ ID NO: 25 - DNA, 18 bp, synthetic construct
  source 1..18: mol_type = other DNA
tcagcctctgcttgatct

SEQ ID NO: 26 - DNA, 23 bp, synthetic construct
  source 1..23: mol_type = other DNA
gcttaatttgactcaacacggga

SEQ ID NO: 27 - DNA, 24 bp, synthetic construct
  source 1..24: mol_type = other DNA
agctatcaatctgtcaatcctgtc

SEQ ID NO: 28 - DNA, 19 bp, synthetic construct
  source 1..19: mol_type = other DNA
ccgtctagaaaaacctgcc

SEQ ID NO: 29 - DNA, 20 bp, synthetic construct
  source 1..20: mol_type = other DNA
gccaaattcgttgtcatacc

(19) United States
(12) Patent Application Publication
(10) Pub. No.: US 2024/0376163 A1
(43) Pub. Date: Nov. 14, 2024

(54) PEPTIDE FOR INHIBITING CALCINEURIN AND USE THEREOF

(71) Applicant: Seoul National University R&DB Foundation, Seoul (KR)

(72) Inventors: Hong Duk YOUN, Gyeonggi-do (KR); Sang Ho LEE, Gyeonggi-do (KR)

(73) Assignee: Seoul National University R&DB Foundation, Seoul (KR)

(21) Appl. No.: 18/660,083

(22) Filed: May 9, 2024

(30) Foreign Application Priority Data
May 10, 2023 (KR) 10-2023-0060717

(51) Int. Cl. C07K 14/47; A61K 38/00

(52) U.S. Cl. CPC C07K 14/47; A61K 38/00

(57) ABSTRACT

The present invention relates a peptide for inhibiting calcineurin and use thereof. The peptide for inhibiting calcineurin, the pharmaceutical composition for immunosuppression including the peptide for inhibiting calcineurin as an active ingredient, the pharmaceutical composition for preventing or treating immune diseases or inflammatory diseases including the peptide for inhibiting calcineurin as an active ingredient, and the health functional food composition for preventing or ameliorating immune diseases or inflammatory diseases of the present invention may specifically inhibit the calcineurin-NFAT pathway, have excellent binding ability to calcineurin, and have excellent in vivo stability. In addition, they may not inhibit the enzymatic activity of calcineurin.

### CROSS-REFERENCE TO RELATED APPLICATION

This application claims priority to and the benefit of Korean Patent Application No. 10-2023-0060717, filed on May 10, 2023, the disclosure of which is incorporated herein by reference in its entirety.

### INCORPORATION BY REFERENCE OF SEQUENCE LISTING

The present application is being filed along with a Sequence Listing in electronic format. The Sequence Listing is provided as a file entitled 717572009900SeqList.xml, created May 9, 2024, which is 26,493 bytes in size. The information in the electronic format of the Sequence Listing is incorporated by reference in its entirety.

### TECHNICAL FIELD

The present invention relates a peptide for inhibiting calcineurin and use thereof.

### BACKGROUND OF THE INVENTION

Representative calcineurin inhibitors widely used around the world include FK506 (Tacrolimus) and Cyclosporin A (CsA). Because these are small molecule drugs, they can be administered orally and have the advantage of being highly effective. In vivo, they bind to FKBP12 and Cyclophilin, respectively, and inhibit the enzymatic activity of calcineurin itself through a mechanism that spatially blocks the enzymatic active site of calcineurin. And they have the function of not only inhibiting the enzymatic activity of calcineurin, but also inhibiting the MAPK pathway and increasing the expression of TGF-beta and several profibrogenic genes. Therefore, if these drugs are administered for a long time, serious side effects such as nephrotoxicity, neurotoxicity, fibrosis, and diabetes appear. In order to avoid these side effects, it is necessary to develop an alternative inhibitor that can specifically block only the calcineurin-NFAT pathway rather than suppress the enzymatic activity of calcineurin.

VIVIT peptide, first developed in 1999, is an artificial peptide inhibitor made based on motifs essential when NFAT binds to calcineurin. It has been reported that this peptide can specifically block the calcineurin-NFAT pathway without inhibiting the enzymatic activity of calcineurin. According to a report published in 2004, when FK506 and VIVIT peptide were treated after mouse pancreatic islet transplantation surgery, insulin reduction, a typical side effect of FK506 treatment, was not observed when VIVIT peptide was treated. This demonstrated the possibility that VIVIT peptide could be an alternative calcineurin inhibitor with fewer side effects.

However, because VIVIT peptide is a protein (peptide)-based compound, it cannot be administered orally, unlike FK506 and CsA, and there is a limitation that the binding ability and inhibitory ability with calcineurin is also significantly low. Therefore, there remains a task of developing an effective drug delivery method and improving binding ability with calcineurin.

The present inventors identified minimal peptides (Pro2146˜Pro2155) essential for binding to calcineurin from CABIN1, an in vivo inhibitory protein of calcineurin, and confirmed that the CABIN1-derived peptide had superior binding ability to calcineurin than the VIVIT peptide to complete the present invention.

### RELATED ART DOCUMENT

### Patent Document

Korean Patent Laid-Open Publication No. 10-2022-0138541 (2022 Oct. 13)

### SUMMARY OF THE INVENTION

### Technical Problem

The present invention is directed to providing a peptide for inhibiting calcineurin, including 8 to 10 consecutive amino acids of the following General Formula 1:

P-X₁-I-X₂-X₃-T-P-P-T-P-T, [General Formula 1]
where in General formula 1, X₁ is valine (V) or glutamic acid (E), X₂ is valine (V) or threonine (T), and X₃ is valine (V) or isoleucine (I).

The present invention is also directed to providing a nucleic acid encoding the peptide.

The present invention is also directed to providing a pharmaceutical composition for immunosuppression including the peptide as an active ingredient.

The present invention is also directed to providing a pharmaceutical composition for preventing or treating immune diseases or inflammatory diseases including the peptide as an active ingredient.

The present invention is also directed to providing a health functional food composition for preventing or ameliorating immune diseases or inflammatory diseases including the peptide as an active ingredient.

Other objects and advantages of the present invention will become clearer from the following detailed description, claims, and drawings.

### Technical Solution

The present invention relates to a peptide for inhibiting calcineurin, including 8 to 10 consecutive amino acids of the following General Formula 1:

P-X₁-I-X₂-X₃-T-P-P-T-P-T, [General Formula 1]
where in General formula 1, X₁ is valine (V) or glutamic acid (E), X₂ is valine (V) or threonine (T), and X₃ is valine (V) or isoleucine (I).

In the present invention, a peptide refers to a molecule formed by combining amino acid residues with one another through an amide bond or peptide bond.

In the present invention, amino acid includes amino acids used to produce peptides, and the amino acid may be alanine (Ala, A), cysteine (Cys, C), aspartic acid (Asp, D), glutamic acid (glu, E), phenylalanine (Phe, F), glycine (Gly, G), histidine (His, H), isoleucine (Ile, I), lysine (Lys, K), leucine (Leu, L), methionine (Met, M), asparagine (Asn, N), proline (Pro, P), glutamine (Gln, Q), arginine (Arg, R), serine (Ser, S), threonine (Thr, T), valine (Val, V), tryptophan (Trp, W), and tyrosine (Tyr, Y).

In the present invention, the peptide may exist in salt form. Salt forms usable in the present invention may be those made during the final isolation and purification of the compound or by reacting the amino group with an appropriate acid. For example, acid addition salts may be acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethanesulfonate, lactate, maleate, mesitylenesulfonate, methanesulfonate, naphthylenesulfonate, nicotinate, 2-naphthalenesulfonate, oxalate, pamoate, pectinate, persulfate, 3-phenylpropionate, picrate, pivalate, propionate, succinate, tartrate, trichloroacetate, trifluoroacetate, phosphate, glutamate, bicarbonate, para-toluenesulfonate, and undecanoate, but are not limited thereto. In addition, examples of acids that can be used to form acid addition salts may be inorganic acids such as hydrochloric acid, hydrobromic acid, sulfuric acid, and phosphoric acid, and organic acids such as oxalic acid, maleic acid, succinic acid, and citric acid, but are not limited thereto.

In the present invention, the consecutive amino acids may include the amino acids of SEQ ID NO: 1. In this case, it may include an amino acid sequence having sequence homology of 70% or more, preferably 80% or more, more preferably 90% or more, and most preferably 95%, 96%, 97%, 98%, 99% or more with the amino acid sequence of SEQ ID NO: 1 (PPTP).

In the present invention, the consecutive amino acids may include the amino acids of SEQ ID NO: 2 or SEQ ID NO: 3. In this case, it may include an amino acid sequence having sequence homology of 70% or more, preferably 80% or more, more preferably 90% or more, and most preferably 95%, 96%, 97%, 98%, 99% or more with the amino acid sequence of SEQ ID NO: 2 (PEITVTPPTP) or SEQ ID NO: 3 (PVIVITPPTP).

In the present invention, the peptide may further include at least one amino acid selected from the group consisting of alanine (A) glycine (G), proline (P) histidine (H), and phenylalanine (F) proline (P) at the N-terminus of the amino acid of General formula 1. In the present invention, CABIN1 peptide refers to a peptide that binds to calcineurin as an in vivo inhibitory protein of calcineurin.

According to an exemplary embodiment of the present invention, to select peptides with improved binding ability to calcineurin, a mammalian two-hybrid technique capable of confirming the binding ability between the two proteins was used to confirm the binding ability between the peptide containing the PxIxIT motif at the C-terminus of CABIN1 and calcineurin. As a result of reducing amino acids one by one before and after the PxIxIT motif, it was confirmed that the peptide (hereinafter, CABIN1-L-1-2), which consists of 10 amino acids from Proline 2146 to Proline 2155, is essential for binding to calcineurin. Compared to the previously known VIVIT peptide, it was confirmed that the “PEITVT” sequence at the N-terminus corresponds to the PxIxIT motif, but the “PPTP” sequence at the C-terminus is different from the existing “GPHEE” sequence, and the “PPTP” sequence plays a very important role in binding to calcineurin. It was confirmed that the CABIN1 peptide is significantly more stable in vivo than the VIVIT peptide and it is because the glutamic acid at the C-terminus of the VIVIT peptide is due to a sequence that repeats twice. In addition, the mechanism by which the “PPTP” sequence of the CABIN1 peptide plays an important role in its binding to calcineurin is as follows. It was confirmed that the phenomenon that CABIN1 peptides have better binding ability to calcineurin than VIVIT peptides is only seen at the cellular level because posttranslational modification (PTM) occurs in CABIN1 peptides, which plays an important role in binding to calcineurin.

In the present invention, the peptide may be one in which threonine (T) residue of the amino acid of General Formula 1 is phosphorylated. Preferably, the threonine (T) may be threonine (T) of SEQ ID NO: 1. According to an exemplary embodiment of the present invention, it was confirmed that Threonine (No. 9 Threonine) residue in the “PPTP” sequence is actually phosphorylated within T cells, and it was demonstrated by performing Surface Plasmon Resonance (SPR) that the phosphorylated CABIN1 peptide is about three times stronger in binding ability to calcineurin compared to the unphosphorylated CABIN1 peptide in vitro.

According to an exemplary embodiment of the present invention, through the above mechanism, it was confirmed that the CABIN1-L-1-2 peptide effectively blocked the calcineurin-NFAT pathway in the Jurkat T cell line, thereby inhibiting the dephosphorylation of NFAT, its migration to the nucleus, and its transcriptional activity more effectively than the VIVIT peptide.

According to an exemplary embodiment of the present invention, in the Jurkat T cell line that was treated with FK506 or expressed CABIN1-L-1-2 peptide, RNA-seq was performed after treating them with a drug that could activate T cells, and it was confirmed that the transcriptome patterns of the two cell lines were similar, and the pathway associated with the immune response was commonly inhibited. Among them, for genes important for the activation of T cells, it was confirmed that CABIN1-L-1-2 peptides suppressed their expression more effectively than VIVIT peptides. Through this, it was demonstrated that CABIN1-L-1-2 peptides can be more effective immunosuppressants by inhibiting the activation of T cells more effectively than VIVIT peptides. In addition, the results that the CABIN1 peptide competes with the VIVIT peptide when it binds to calcineurin confirmed that it binds to the same position as the VIVIT peptide. As with the previously known characteristics of the VIVIT peptide, it was confirmed that the binding of the CABIN1 peptide at this position did not inhibit the enzymatic activity of calcineurin. Through this, it was confirmed that the CABIN1 peptide is likely to have the same advantages of the VIVIT peptide (which does not show insulin reduction side effects when administered with FK506).

In addition, the present invention relates to a nucleic acid encoding the peptide for inhibiting calcineurin.

In the present invention, nucleic acid refers to a polymer organic material in which nucleotides are polymerized in a long chain shape.

In the present invention, the phosphorylation may be induced by p38MAPK. According to an exemplary embodiment of the present invention, it was confirmed phosphorylation of the CABIN1 peptide is caused by p38MAPK in vivo.

In the present invention, the phosphorylation may occur within T cells.

In the present invention, the peptide may inhibit the proliferation of T cells.

In the present invention, the peptide may be derived from CABIN1 peptide.

In the present invention, the peptide may inhibit CN-NFAT pathway (Calcineurin-NFAT pathway). The CN-NFAT pathway is very important for the activation of T cells. Thus, the peptide may be used as an immunosuppressant by inhibiting the activation of T cells, and since the CN-NFAT pathway plays an important role in various cancer cells such as pancreatic and colon cancers, the peptide may be used as an anticancer agent.

The present invention relates to a pharmaceutical composition for immunosuppression including the peptide for inhibiting calcineurin as an active ingredient.

In the present invention, the active ingredient refers to a component capable of exhibiting desired activity alone or exhibiting activity together with a carrier having no activity itself.

When the composition according to the present invention is in the form of a pharmaceutical composition, the composition may include a pharmaceutically effective amount of peptide alone or may include one or more pharmaceutically acceptable carriers. In this case, the pharmaceutically acceptable carrier is one commonly used in preparation, and includes, but is not limited to, lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate, and mineral oil, etc. In addition, it may further include lubricant, wetting agent, sweetening agent, flavoring agent, emulsifying agent, suspending agent, preservative, etc. in addition to the above components.

The pharmaceutical composition of the present invention may be administered orally or parenterally (e.g., applied intravenously, subcutaneously, intraperitoneally or locally) depending on the desired method, and the dosage varies depending on the patient's condition and weight, degree of disease, drug type, administration route, and time, but may be appropriately selected by those skilled in the art.

The pharmaceutical composition of the present invention is administered in a pharmaceutically effective amount. In the present invention, the “pharmaceutically effective amount” means an amount sufficient to treat a disease with a reasonable benefit/risk ratio applicable to medical treatment, and the effective dose level may be determined according to the patient's disease type, severity, drug activity, drug sensitivity, administration time, administration route and excretion rate, duration of treatment, factors including concomitant drugs, and other factors well known in the medical field. The pharmaceutical composition according to the present invention may be administered as an individual therapeutic agent or in combination with other therapeutic agents, may be administered sequentially or simultaneously with conventional therapeutic agents, and may be administered single or multiple. In consideration of all of the above factors, it is important to administer an amount that can obtain the maximum effect with a minimum amount without side effects, which can be easily determined by those skilled in the art.

Specifically, the effective amount of the pharmaceutical composition of the present invention may vary depending on the patient's age, sex, condition, weight, absorption of active ingredients in the body, inactivity rate and excretion rate, disease type, and drug used in combination, and generally, 1 to 500 mg per kg of body weight may be administered daily or every other day, or it may be administered in 1 to 3 divided doses per day. However, since it may increase or decrease depending on the route of administration, sex, weight, age, etc., the dosage is not intended to limit the scope of the present invention in any way.

The present invention relates to a pharmaceutical composition for preventing or treating immune diseases or inflammatory diseases including the peptide for inhibiting calcineurin as an active ingredient.

In the present invention, the immune disease may be at least one selected from the group consisting of: rheumatoid arthritis, Bechcet's disease, multiple myositis or skin myositis, autoimmune hemocytopenia, autoimmune myocarditis, atopic dermatitis, asthma, primary cirrhosis, dermatomyositis, Goodpasture's syndrome, autoimmune meningitis, Sjogren's syndrome, lupus, Addison's disease, alopecia areata, ankylosing spondylitis, autoimmune hepatitis, autoimmune parotitis, Crohn's disease, insulin-dependent diabetes, dystrophic epidermolysis bullosa, epididymitis, glomerulonephritis, Graves' disease, Guillain-Barré syndrome, Hashimoto's disease, hemolytic anemia, multiple sclerosis, myasthenia gravis, pemphigus vulgaris, psoriasis, rheumatic fever, sarcoidosis, scleroderma, spondyloarthropathy, thyroiditis, vasculitis, vitiligo, myxedema, pernicious anemia, mitochondria-related syndrome, and ulcerative colitis.

In the present invention, the inflammatory disease may be at least one selected from the group consisting of: edema, allergy, periodontitis, rhinitis, otitis media, sore throat, tonsillitis, pneumonia, gastric ulcer, gastritis, colitis, hemorrhoids, gout, ankylosing spondylitis, arthritis, osteoarthritis, periarteritis, tendinitis, tenosynovitis, myositis, hepatitis, cystitis and nephritis.

The present invention relates to a health functional food composition for preventing or ameliorating immune diseases or inflammatory diseases including the peptide for inhibiting calcineurin as an active ingredient.

When the composition according to the present invention is in the form of a health functional food composition, in addition to certain health foods and nutritional supplies, it can be prepared with medical and medical-effective foods that are processed to have an efficient bioregulatory function, and in some cases, the food may be used interchangeably as a functional food, a health food, or a health supplement, and may be prepared in various forms such as tablets, capsules, powders, granules, liquids, and pills to obtain useful effects.

The health functional food of the present invention may contain additional ingredients that are commonly used in food compositions to improve smell, taste, vision, etc. For example, it may include vitamins A, C, D, E, B1, B2, B6, B12, niacin, biotin, folate, panthothenic acid, etc. In addition, it may include minerals such as zinc (Zn), iron (Fe), calcium (Ca), chromium (Cr), magnesium (Mg), manganese (Mn), and copper (Cu). In addition, it may include amino acids such as lysine, tryptophan, cysteine, and valine. In addition, food additives such as preservatives (potassium sorbate, sodium benzoate, salicylic acid, sodium dihydroacetate, etc.), disinfectants (bleaching powder and advanced bleaching powder, sodium hypochlorite, etc.), antioxidants (butylhydroxyanisole (BHA), butylhydroxytoluene (BHT), etc.), colorants (tar pigments, etc.), color developers (sodium nitrite, sodium acetate, etc.), bleach (sodium sulfite), seasoning (sodium MSG glutamate, etc.), sweeteners (dulsin, cyclamate, saccharin, sodium, etc.), flavorings (vanillin, lactone, etc.), expansion agents (alumn, D-potassium hydrogen tartarate, etc.), reinforcement agents, emulsifying agents, thickeners, coating agents, gum base agents, foam inhibitors, solvents, and improvers can be added. The additive may be selected according to the type of food and used in an appropriate amount.

When the health functional food of the present invention is used as a food additive, it may be added as it is or used together with other food or food ingredients, and may be appropriately used according to a conventional method.

In the health functional food of the present invention, the content of peptides is not particularly limited, and may be variously changed depending on the condition of the subject to be administered, the specific type of disease, and the degree of progression, etc. If necessary, it may also be included as the total content of the food.

### Advantageous Effects

The peptide for inhibiting calcineurin, the pharmaceutical composition for immunosuppression including the peptide for inhibiting calcineurin as an active ingredient, the pharmaceutical composition for preventing or treating immune diseases or inflammatory diseases including the peptide for inhibiting calcineurin as an active ingredient, and the health functional food composition for preventing or ameliorating immune diseases or inflammatory diseases of the present invention may specifically inhibit the calcineurin-NFAT pathway, have excellent binding ability to calcineurin, and have excellent in vivo stability. In addition, they may not inhibit the enzymatic activity of calcineurin.

### BRIEF DESCRIPTION OF THE DRAWINGS

FIGS. 1A-1P show the interaction between peptides and calcineurin. FIGS. 1A to 1D show that a short peptide at the CABIN1 C-terminus is essential for interaction with calcineurin. FIG. 1A is a schematic diagram and sequence of CABIN1 fragments. FIGS. 1B and 1C show the results of mammalian two-hybrid assay using VP16 AD-CNAB2 and GAL4 DBD-CABIN1 peptides (b: CABIN1-14,-15, -S to L; c: CABIN1-L-1 to L-1-4) in HEK293T cells. FIG. 1D shows sequences (top) of CABIN1-L-1-2, VIVIT 10-mer, and chimeric peptides. A mammalian two-hybrid assay using these peptides was performed (bottom). The construction of transfected DNA is described in FIGS. 1E to 1G. All experiments were repeated three times and are expressed as mean±standard deviation. The expression level of peptide was confirmed by Western blotting. SE: short exposure, LE: long exposure, NC: negative control, WT: wild type. FIGS. 1E to 1I show data on the interaction between CNA and CABIN1 fragments. FIGS. 1E to 1G are compositions of transfected DNA for the mammalian two-hybrid assay in FIGS. 1B to 1D, respectively. FIGS. 1H and 1I show the results of transfection of HEK293T cells with Flag-CNAβ2 and GAL4 DBD-CABIN1 fragments and immunoprecipitation with Flag antibody. Interactions between CNAβ2 and each CABIN1 fragment were detected with the indicated antibodies. SE: short exposure, LE: long exposure, NC: negative control, WT: wild type. FIGS. 1J to 1L show the role of adjacent sequences around the “PxIxIT” motif. FIG. 1J is a sequence of CABIN1, VIVIT, and chimeric peptides. FIG. 1K shows the results of immunoprecipitation assay using Flag-CNAβ2 and GST-peptides in HEK293T. FIG. 1L shows the results of measuring NFAT transcriptional activity in Jurkat T cells expressing GST-tagged peptides under PMA and ionomycin treatment by performing a luciferase reporter assay. All experiments were repeated three times and expressed as mean±standard deviation. The expression level of peptide was confirmed by Western blotting. NC: negative control. FIGS. 1M to 1P show the protein stability of the VIVIT peptide. FIG. 1M is a sequence of the VIVIT peptide. In FIG. 1N, the protein expression level of the VIVIT peptide was detected with HA antibodies in Jurkat T cells expressing HA-mCherry-tagged VIVIT peptides. In FIG. 1O, the mCherry expression intensity and distribution were analyzed by FACS in Jurkat T cells expressing HA-mCherry-tagged VIVIT peptides. (5×10⁴ cells/sample). In FIG. 1P, the mRNA expression level of mCherry was measured by qRT-PCR. The values shown are mean±standard deviation (n=4). SE: short exposure, LE: long exposure, NC: negative control.

FIGS. 2A-2E show that the CABIN1 peptide inhibits the CN-NFAT pathway more efficiently than the VIVIT peptide. In FIG. 2A, a luciferase reporter assay was performed to measure NFAT transcriptional activity. Jurkat T cells expressing GST-tagged peptides were treated with PMA and ionomycin for activation. Jurkat T cells expressing only GST were used as negative controls, and cells treated with 0.5 μM FK506 were used as positive controls (n=3). In FIG. 2B, an NFAT mobility shifting assay was performed using Jurkat T cells expressing HA-mCherry-tagged peptides. For dephosphorylation of NFATC2, Jurkat T cells were treated with PMA and ionomycin for 2 hours. In FIG. 2C, the hyperphosphorylated and hypophosphorylated NFATC2 of FIG. 2B was measured using ImageJ, and the hypo/hyper ratio was calculated (n=7). In FIG. 2D, snapshot images of the indicated peptides and activated Jurkat T cells expressing tNFATC2 were obtained using a confocal microscope. Expression and intracellular localization of peptides and tNFATC2 are indicated by mCherry (red) and EGFP (green), respectively. The nucleus was stained with Hoechst (blue). A scale bar, 50 μm. In FIG. 2E, tNFATC2 located in the nucleus was measured by calculating a ratio at which Hoechst (M1) and EGFP (M2) were present together. (NC and FK506, n=10; CABIN1-L-1-2 and VIVIT 10-mer, n=12). The data were repeated independently as many times as described and are expressed as means±standard deviation. Statistical differences were determined using two-sided Student's t-test (A, C) or one-way analysis of variance (E). *** p<0.001, NC: negative control.

FIGS. 3A-3D show that CABIN1 peptide inhibits T cell activation. FIG. 3A is a heatmap of genes whose transcription amount was changed by FK506 treatment or CABIN1 peptide expression during T cell activation. FIG. 3B is gene ontology analysis of cluster III. FIG. 3C is the mRNA expression level of genes included in cluster III. FIG. 3D is qRT-PCR data of the genes in FIG. 3C. The experiment was independently repeated at least three times, and data were expressed as mean±standard deviation. Statistical differences were determined using a two-sided Student's t-test. * p<0.05, ** p<0.01, *** p<0.001, NC: negative control.

FIGS. 4A-4D show that CABIN1 peptide does not affect the phosphatase activity of calcineurin. In FIG. 4A, an in vitro peptide competition assay was performed using VIVIT 15-mer and CABIN1-L-1 peptide in a dose-dependent manner. The molar ratio of GST-tagged peptide to competing peptide ranged from 1:50 to 1:400. In FIG. 4B, a mammalian two-hybrid assay was performed using VP16 AD-CNAβ2 and GAL4 DBD-peptide in HEK293T cells treated or not-treated with 1 μM ionomycin. The pM empty vector was used as a negative control. In FIG. 4C, a calcineurin cellular activity assay was performed using Jurkat T cells expressing HA-mCherry-tagged peptides. Jurkat T cells expressing only HA-mCherry were used as negative controls, and cells treated with FK506 were used as positive controls. FIG. 4D shows the results of in vitro calcineurin phosphatase activity analysis using purified GST-tagged peptides. Purified GST was used as a negative control. All experiments were repeated three times, and data were expressed as mean±standard deviation. Statistical differences were determined using one-way analysis of variance. * p<0.05, NS: no statistical significance, NC: negative control.

FIGS. 5A-5C show that PPTP sequences are important for interaction with calcineurin at the cellular level. FIG. 5A shows the results of immunoprecipitation assay (left) using the indicated tagged CNAβ2 and peptides in HEK293T cells, and in vitro His-pull down assay (right) using purified protein. In FIG. 5B, a mammalian two-hybrid assay was performed using CNAβ2 and substituted CABIN1 peptides to confirm binding affinity (n=3). HEK293T cells were transfected as described in the right panel. FIG. 5C shows the results of immunoprecipitation assay (left) using the indicated tagged CNAβ2 and peptides in HEK293T cells, and in vitro His-pull down assay (right) using purified protein. The values shown are mean±standard deviation. NC: negative control, WT: wild type.

FIGS. 6A-6C show that hydroxylation or O-GlcNAcylation does not occur in the “PPTP” sequence. In FIG. 6A, Jurkat T cells expressing HA-mCherry-tagged CABIN1 peptide were treated with DMOG for 12 hours in a dose-dependent manner. In FIG. 6B, Jurkat T cells expressing HA-mCherry-tagged CABIN1 peptide were treated with 500 μM DMOG over time. Each cell lysate was immunoprecipitated with HA antibodies, and CNA bound to CABIN1 peptides was detected by Western blotting. HIF-1α was used as a positive control for DMOG treatment. In FIG. 6C, HEK293T cells were transfected with GST-tagged peptides and subjected to GST pull-down assay. Hydroxyproline and O-linked glycosylation of each peptide were detected by Western blotting. NC: negative control.

FIGS. 7A-7G shows that phosphorylation of CABIN1 T2154 is important for binding to calcineurin. FIG. 7A is the sequence of the CABIN1 peptide with a threonine to valine substitution (TV). FIG. 7B shows the results of immunoprecipitation assay (top) with the indicated tagged CNAβ2 and wild-type or TV CABIN1 peptides in HEK293T cells, and in vitro His-pull down assay (bottom) using purified protein. In FIG. 7C, a luciferase reporter assay was performed to measure NFAT transcriptional activity in Jurkat T cells expressing GST-tagged CABIN1 peptides under PMA and ionomycin treatment (n=3). FIG. 7D shows the results of immunoprecipitation assay of Jurkat T cells expressing HA-mCherry-tagged peptides using HA antibodies. CNA bound to the peptide and phosphorylated threonine were detected by Western blotting. In FIG. 7E, phosphorylation of the 9th threonine of the CABIN1 peptide (threonine 2154 of full-length CABIN1) was detected by LC-MS/MS in Jurkat T cells. In FIG. 7F, an in vitro pull-down assay was performed using streptavidin-coupled agarose beads. CNA bound to the biotinylated peptide was detected with a His antibody. VEET peptide was used as a negative control. In FIG. 7G, CNA binding to biotinylated CABIN1 peptide according to the presence or absence of phosphorylation of the 9th threonine was measured by SPR. CNA concentration ranges are 1.25, 2.5, 5, and 10 nM. The kinetics of the interaction were calculated using the data of the CABIN1 peptide-captured cells minus the negative control peptide-captured cell. The values shown are mean±standard deviation. RU: reaction unit, NC: negative control.

FIGS. 8A-8H show that p38MAPK phosphorylates the 9th threonine of CABIN1 peptide. In FIG. 8A, Jurkat T cells expressing HA-mCherry-tagged CABIN1 peptide were treated with 40 nM PMA in a time-dependent manner. Phosphorylated threonine in peptides immunoprecipitated with HA antibody was detected with [PXpTP] antibody. FIG. 8B shows the results of in vitro kinase assay using purified GST-tagged CABIN1 peptide and PKC downstream kinase. FIG. 8C shows the results of in vitro kinase assay using kinases included in the MAPK family and purified GST-tagged peptides. FIG. 8D shows the results of immunoprecipitation assay using Flag-MAPK and HA-CABIN1 in HEK293T cells. FIGS. 8E and 8F show the results of in vitro kinase assay using p38 and substituted CABIN1 peptide. In FIG. 8G, Jurkat T cells expressing HA-mCherry-tagged CABIN1 peptide were treated with 10 μM p38a inhibitor for 16 hours. Immunoprecipitation was performed with HA antibody and detected by Western blotting. In FIG. 8H, phosphorylated threonine and peptide-bound CNA in FIG. 8G were measured using ImageJ, and the ratio of phosphorylation and calcineurin bound to CABIN1 peptide was calculated (n=6). The values shown are mean±standard deviation. Statistical differences were determined using one-way analysis of variance. * p<0.05, ** p<0.01, *** p<0.001, CBB: Coomassie Brilliant Blue, NC: negative control.

FIGS. 9A and 9B show putative calcineurin target genes that are not regulated through the calcineurin-NFAT pathway. FIG. 9A shows the results of mRNA expression levels of genes suppressed by FK506 but not affected by CABIN1 peptide. FIG. 9B is qRT-PCR data of the genes shown in FIG. 9A. The experiment was independently repeated at least three times, and expressed as mean±standard deviation. Statistical differences were determined using one-way analysis of variance. *** p<0.001, NS: no statistical significance, NC: negative control.

### DETAILED DESCRIPTION OF THE EMBODIMENTS

Hereinafter, in order to help understanding of the present invention, examples will be described in detail. However, the following examples are only intended to illustrate the content of the present invention, and the scope of the present invention is not limited to the following examples. Examples of the present invention are provided to describe the present invention more fully to those skilled in the art.

### Example 1. Experimental Materials and Methods

### 1.1 Cell Culture and Transfection

HEK293T and Jurkat T (E6-1) cells were purchased from ATCC (Manassas, VA, USA). HEK293T cells were cultured at 37° C. and 5% CO2 in Dulbecco's modified Eagle's medium supplemented with 10% (v/v) fetal bovine serum (Gibco, Grand Island, NY, USA) and antibiotics and transfected using polyethyleneimine (Polysciences, Warrington, FL, USA). Jurkat T cells were cultured at 37° C. and 5% CO2 in RPMI 1640 medium supplemented with 10% (v/v) fetal bovine serum (Gibco), 2 mM L-glutamine (Gibco), and antibiotics. Jurkat T cells were transfected by electroporation using a Neon™ Transfection System (Thermo Fisher Scientific, Waltham, MA, USA) according to the manufacturer's instructions.

### 1.2 Plasmid DNA

The calcineurin catalytic subunit (CNA), isoform, CABIN1-14, CABIN1-15, INFATC2, pG5-luc, and NFAT-luc DNA were used in previous reports (Sun, L. et al., Immunity 8, 703-711 (1998)). CNA isoform was cloned into expression vectors such as pVP16, pRSET-A/B, and pCAG-Flag. Oligonucleotides and chimeric and substituted peptides of CABIN1, VIVIT were obtained from Macrogen (Seoul, Korea) or Cosmogenetech (Seoul, Korea), annealed for hybridization and inserted into expression vectors such as pM, pGEX4T-1, pCAG-HA-GST, and pCAG-HA-mCherry. The pCAG-EGFP-tNFATC2-IRES-mCherry-peptide was constructed by replacing the puromycin resistance gene following the IRES segment with an mCherry-tagged peptide. All structures were confirmed by sequencing.

### 1.3 Reagents and Antibodies

Anti-GAL4 DNA binding domain (GAL4 DBD) (RK5C1) antibodies from Santa Cruz Biotechnology (Dallas, TX, USA) and anti-NFATC2, pan-CNA, and phospho-MAPK Substrates Motif [PXpTP] antibodies from Cell Signaling (Danvers, MA, USA) were purchased. Anti-β-actin (AC-15) and Flag (M2) antibodies and Anti-FLAG® M2 Affinity Gel were purchased from Sigma-Aldrich (St. Louis, MO, USA). Anti-HA (16B12) antibody was obtained from BioLegend (San Diego, CA, USA) and Pierce™ Anti-HA Agarose from Thermo Fisher Scientific. PMA (phorbol myristate acetate) and ionomycin from Sigma-Aldrich, FK506 from InvivoGen (San Diego, CA, USA), and EO 1428 and TAK 715 from Tocris (Bristol, UK) were purchased.

### 1.4 Peptide Synthesis

Peptron (Daejeon, Korea) synthesized unlabeled peptides (CABIN1-L-1 [MAGFPPEITVTPPTP] and VIVIT 15-mer [MAGPHPVIVITGPHEE]). GenScript (Piscataway, NJ, USA) synthesized biotin-labeled peptides (VEET [Biotin-AAAMAGPPHIVEETGPHVI], VIVIT 15-mer [Biotin-AAAMAGPHPVIVITGPHEE], CABIN1-L-1 [Biotin-AAAMAGFPPEITVTPPTP] and phosphorylated CABIN1-L-1 [Biotin-AAAMAGFPPEITVTPP {pTHR} P]). All peptides were purified by HPLC and were more than 95% (>95%) pure, and their molecular weight and composition were analyzed by mass spectrometry.

### 1.5 Luciferase Reporter Assay

To measure the transcriptional activity of NFAT, Jurkat T cells were cotransfected with GST-tagged peptides and NFAT-luc by electroporation. After 24 hours, the cells were pretreated with 0.5 μM FK506 for 1 hour and then treated with 40 nM PMA and 1 μM ionomycin for 8 hours. For mammalian two-hybrid assay, HEK293T cells were cotransfected with GAL4 DBD-tagged CABIN1, VIVIT, chimeric, and substituted peptides, VP16 activation domain-tagged CNAβ2, and pG5-luc (a luciferase reporter plasmid containing the GAL4 reaction promoter) for 24 hours. Harvested cells were lysed with luciferase assay lysis buffer (17 mM KH₂PO₄, 183 mM K₂HPO₄, pH 7.8, 0.2% [v/v] Triton X-100) and sonicated briefly. The luciferase activity against each cell lysate was measured using Infinite M200 (Tecan, Männedorf, and Switzerland). In addition, the expression level of the transfected protein was confirmed by Western blotting.

### 1.6 Immunoprecipitation Assay

Cells were lysed in IP150 buffer and immunoprecipitated Flag- and HA-tagged proteins using Anti-FLAG® M2 Affinity Gel (Sigma-Aldrich) and Pierce™ Anti-HA Agarose (Thermo Fisher Scientific), respectively. Conventional methods were used to construct the buffer and subsequent processes (Kim, H. J. et al. Nuclear Acids Res. 46, 6544-6560 (2018)). To quantify the Western blotting results, band regions were measured using ImageJ (https://imagej.nih.gov/ij/).

### 1.7 Protein Purification and In Vitro Binding Assay

GST-tagged peptide and His-tagged CNAα and β2 were expressed in Escherichia coli BL21 (DE3) pLysS. When the OD₆₀₀ reached 0.5, the bacteria were treated with IPTG (0.1 mM overnight at 18° C. or 1 mM for 4 hours at 37° C.). Proteins were purified in the conventional method (Lee, H. T. et al. Cancers 13, 3418 (2021)). A His-pulldown assay was performed with 0.5 ml of IP150 buffer containing 0.5 mM EDTA. His-tagged CNA and GST-tagged peptides were mixed and captured on Ni-NTA agarose beads (Qiagen, Hilden, Germany). The beads were washed three times with binding buffer, and the proteins were analyzed by Western blotting. GST-tagged peptides were pulled down using Glutathione Sepharose™ 4 Fast Flow (Cytiva, Marlborough, MA, USA), and biotin-labeled peptides were pulled down using Streptavidin Agarose Resin (Thermo Fisher Scientific).

### 1.8 In Vitro Kinase Assay

Purified GST-tagged peptides were used for the in vitro kinase assay using activated recombinant kinase obtained from Proqinase GmbH (Freiburg, Germany). Kinase reactions were induced in reaction buffer (50 mM HEPES-NaOH, pH 7.5, 3 mM MgCl₂, 3 mM MnCl₂, 3 μM sodium orthobanadate, 1 mM dithiothreitol, 50 μM unlabeled ATP, 5 μCi 32P-γ-ATP) for 1 hour at 30° C. The reaction was stopped by the addition of 5× sample buffer, and proteins were isolated using SDS-PAGE. The gels were stained with Coomassie Brilliant Blue, dried, and developed on X-ray film.

### 1.9 Confocal Microscopy and Analysis

Jurkat T cells were transfected with pCAG-EGFP-tNFATC2-IRES-mCherry-peptide by electroporation. Two days later, these cells were pretreated with 0.5 M FK506 for 30 min. Afterwards, they were treated with 40 nM PMA and 1 μM ionomycin for 4 hours under culture conditions. Nuclei were stained by treating these cells with 10 μg/ml Hoechst 33342 (Invitrogen, Waltham, MA, USA) before imaging with a confocal laser scanning microscopy (Nikon, Tokyo, Japan). Colocalization analysis of mCherry and Hoechst was performed with ImageJ and corresponding plug-in JACoP to measure the Manders coefficient (Manders, E. M. et al., J. Cell Sci. 103, 857-862 (1992), Bolte, J. Microsc. 224, 213-232 (2006)).

### 1.10 Calcineurin Activity Assay

Calcineurin phosphatase activity was measured in vitro using the Calcineurin phosphatase assay kit (BML-AK804, Enzo Life Sciences, Farmingdale, NY, USA). GST or GST-tagged peptides were cultured with CNA for 30 min at room temperature and then performed according to the manufacturer's instructions. Jurkat T cells were transiently transfected with CAG-HA-mCherry-peptide by electroporation endogenous calcineurin phosphatase activity. After 24 hours, the Jurkat T cells were pretreated with FK506 for 1 hour before activation with PMA and ionomycin. Thereafter, the cells were washed with TBS buffer (20 mM Tris, pH 7.2, 150 mM NaCl), resuspended with lysis buffer in a calcineurin cell activity assay kit (BML-AK816, Enzo Life Sciences), and stored on ice for 30 min. Lysate supernatants were collected by gel filtration using Desaltting Column Resin included in the kit, free phosphate was removed, and performed according to the manufacturer's instructions.

### 1.11 Real-Time Quantitative PCR

Jurkat T cells were collected by QIAzol Lysis Reagent (Qiagen) to extract total RNA. cDNA was reverse transcribed from the isolated RNA using AMV Reverse Transcriptase and random hexamers. Real-time quantitative PCR for each cDNA was performed using TB Green® Premix Ex Taq™ (TaKaRa, Kusatsu, Japan) in the CFX Connect Real-time PCR Detection System (Bio-Rad, Hercules, CA, USA), and normalized to expression of 18S rRNA or GAPDH. Table 1 shows the primers used.

TABLE 1

| | Forward | Reverse |
|---|---|---|
| Gene | Primer Sequence | Primer Sequence |
| IL2 | ACAGCTACAACTGGAGCA | TCAGTTCTGTGGCCTTC |
| | TTTA (SEQ ID NO: 4) | TTG (SEQ ID NO: 5) |
| IL3 | GCTGGACTTCAACAACCT | CAGACATGGCAGGAGATT |
| | (SEQ ID NO: 6) | T (SEQ ID NO: 7) |
| CCL4 | GTACGTGTATGACCTGGA | GAGATGTGTCTCATGGAG |
| | AC (SEQ ID NO: 8) | AAG (SEQ ID NO: 9) |
| CCL20 | GACTGCTGTCTTGGATAC | TACTGAGGAGACGCACAA |
| | AC (SEQ ID NO: 10) | (SEQ ID NO: 11) |
| CXCL8 | GAACCATCTCACTGTGTG | TGGAAAGGTTTGGAGTAT |
| | TAA (SEQ ID NO: 12) | GTC (SEQ ID NO: 13) |
| NFATC1 | CCGTTCACGTCAGTTTCT | GTTGGAGCAGGCTCATAA |
| | AC (SEQ ID NO: 14) | TC (SEQ ID NO: 15) |
| CD27 | GCACTGTAACTCTGGTCT | GGGTTTGGAAGAGGATCA |
| | TC (SEQ ID NO: 16) | C (SEQ ID NO: 17) |
| CD70 | GATGGCATCTACATGGTA | GGTACAACCTTGGTGGAA |
| | CAC (SEQ ID NO: 18) | G (SEQ ID NO: 19) |
| NINJ1 | CCTCATCTCCATCTCCCT | GTTGACTACCACGATGAT |
| | T (SEQ ID NO: 20) | GAA (SEQ ID NO: 21) |
| ZP4 | GGAGACCGAGCAGTATAT | CTGGACATTGATTGGGAG |
| | GA (SEQ ID NO: 22) | AG (SEQ ID NO: 23) |
| mCherry | ACGGCGAGTTCATCTACA | TCAGCCTCTGCTTGATCT |
| | (SEQ ID NO: 24) | (SEQ ID NO: 25) |
| 18S | GCTTAATTTGACTCAACA | AGCTATCAATCTGTCAAT |
| rRNA | CGGGA (SEQ ID | CCTGTC (SEQ ID |
| | NO: 26) | NO: 27) |
| GAPDH | CCGTCTAGAAAAACCTGC | GCCAAATTCGTTGTCATA |
| | C (SEQ ID NO: 28) | CC (SEQ ID NO: 29) |

1.12 mRNA Sequencing and Analysis

Jurkat T cells expressing HA-mCherry-tagged peptides were pretreated with FK506 for 1 hour and then activated with PMA and ionomycin for 8 hours. Thereafter, RNA was extracted from these cells. cDNA library construction and next-generation sequencing were performed at LAS (Gimpo, Korea). All samples were sequenced with 75 paired-end reads on an Illumina NextSeq 500 system. For RNA sequencing analysis, readings for each sample were aligned to the human genome (GRCh37/hg19 genome assembly) using STAR 2.4.0.125 as the default setting. The HOMER tool was used to quantify and normalize the defined genes in the RefSeq transcriptome. FPKM normalized genes were clustered hierarchically using Cluster3.0 (de Hoon, M. J. et al., Bioinformatics 20, 1453-1454 (2004)). Refined data were visualized with Java Treeview (http://jtreeview.sourceforge.net). Gene ontology analysis of each cluster was performed using the DAVID Web tool (https://david.ncifcrf.gov). Finally, expression levels of target genes were visualized using the Integrative Genomics Viewer (http://software.broadinstitute.org/software/igv). Raw data were submitted to NCBI Gene Expression Omnibus under accession number GSE185561.

### 1.13 Flow Cytometry

LSRII (SORP) (Becton Dickinson, Franklin Lakes, NJ, USA) was used to quantify the expression of mCherry-tagged VIVIT peptides. Results were analyzed with FlowJo (Tree Star, Inc., Ashland, OR, USA).

### 1.14 Mass Spectrometry

HA-mCherry-tagged CABIN1-L-1-2 peptides expressed in Jurkat T cells were purified by immunoprecipitation of 4 mg of lysate with Pierce™ Anti-HA Agarose. The bead-binding protein was eluted by competing with 1 mg/ml HA peptide. Purified proteins were analyzed by LC-MS/MS (ProteomeTech, Inc., Seoul, Korea) after separation by SDS-PAGE and staining with Coomassie Brilliant Blue.

### 1.15 Surface Plasmon Resonance

Biacore T200 (Cytiva) was used to capture biotin-labeled peptides on the surface of Series S Sensor Chip SA (Cytiva). Purified His-tagged CNA in HBS-EP+ (10 mM HEPES, pH 7.4, 0.15 M NaCl, 3 mM EDTA, 0.05% surfactant P20) was serially diluted and passed through three flow cells (flow cell 1: biotin-VEET, flow cell 2: biotin-CABIN1-L-1, flow cell 3: biotin-p-CABIN1-L-1). Each cycle was performed at 25° C. with 120 seconds of contact and 240 seconds of dissociation at a flow rate of 30 μl/min. The kinetics of the interaction were analyzed using BIAvaluation 3.2 RC1 (Cytiva).

### 1.16 Statistics

All data are expressed as mean±standard deviation obtained from at least three biologically independent tests. The p-values were calculated using two-sided Student's t-test to compare two groups and two-sided one-way ANOVA with Tukey's multiple comparison test to analyze multiple groups. The p-values<0.05, <0.01, and <0.001 are indicated by single, double, and triple asterisks, respectively. NS indicates non-significant difference (p≥0.05).

### Example 2. Experiment Result

2.1 Ten Amino Acids of CABIN1 (2146-2155) Essential for Interaction with Calcineurin

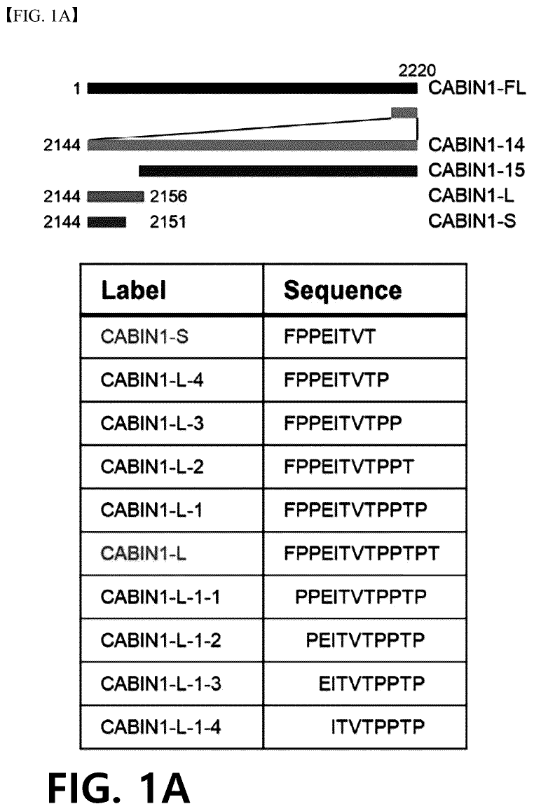

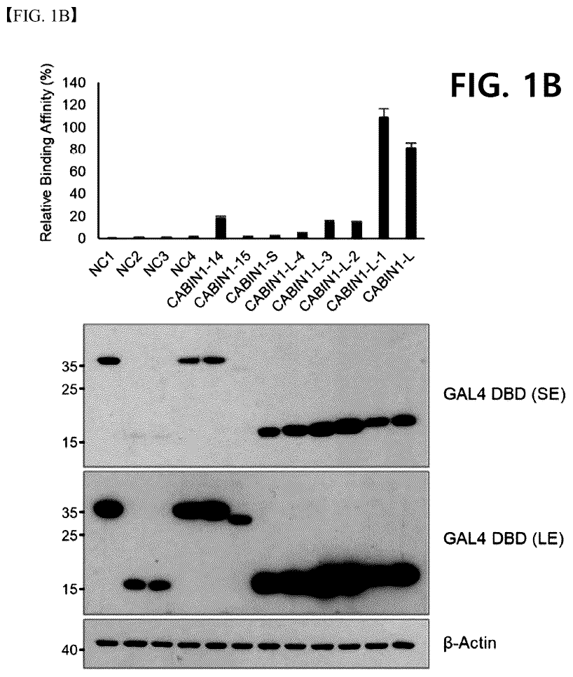

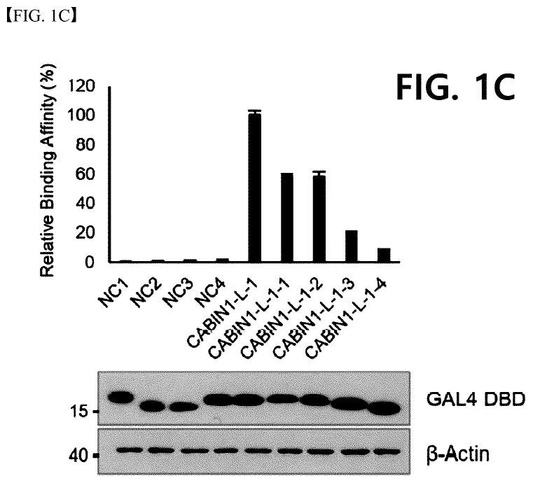

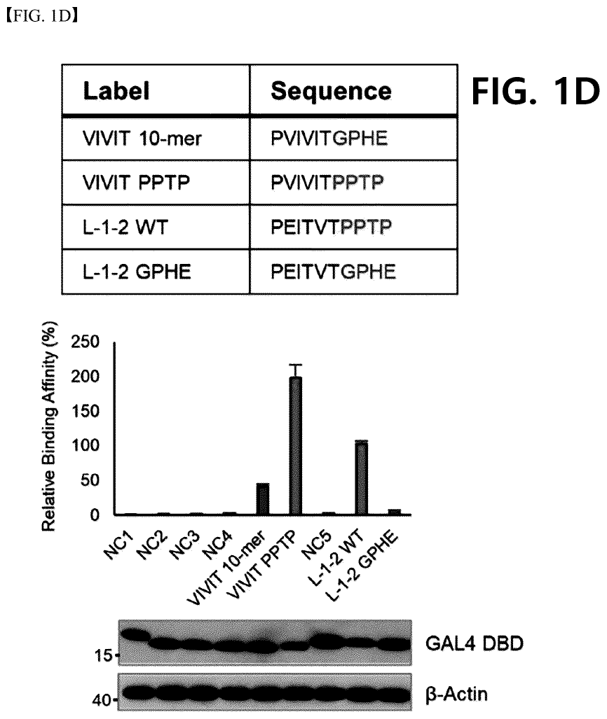

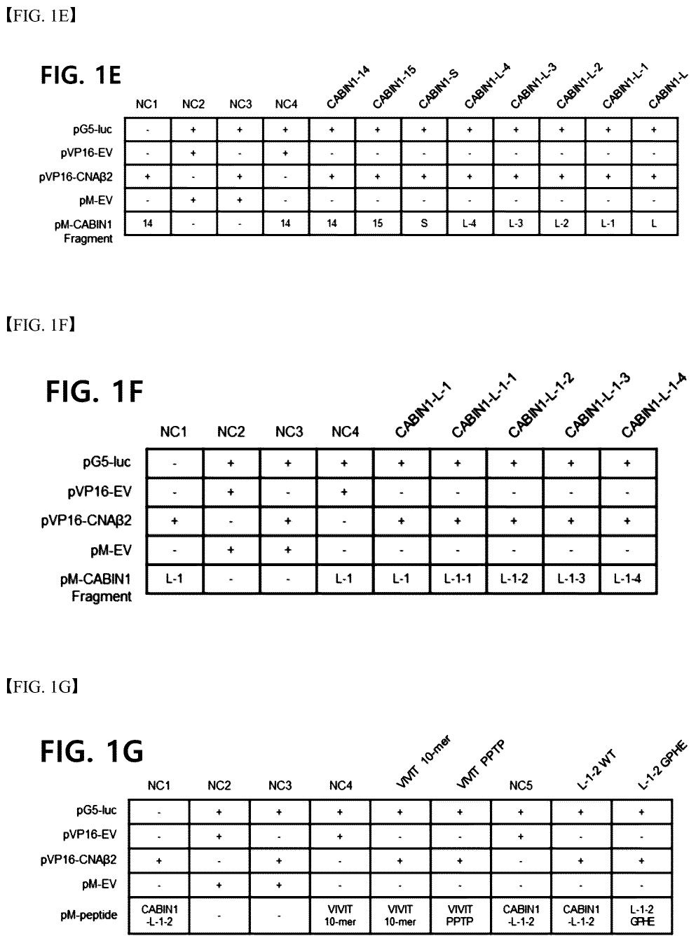

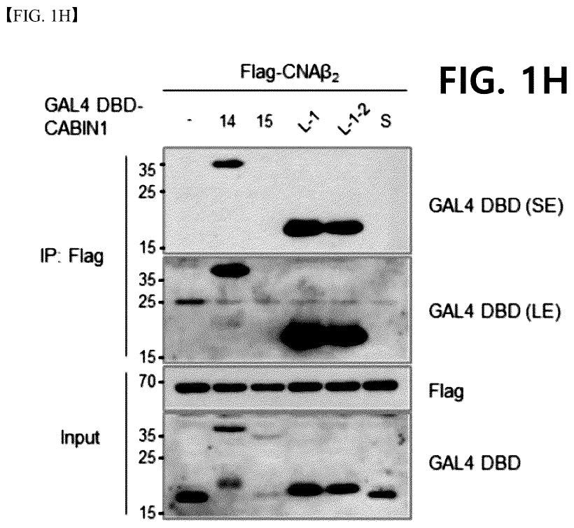

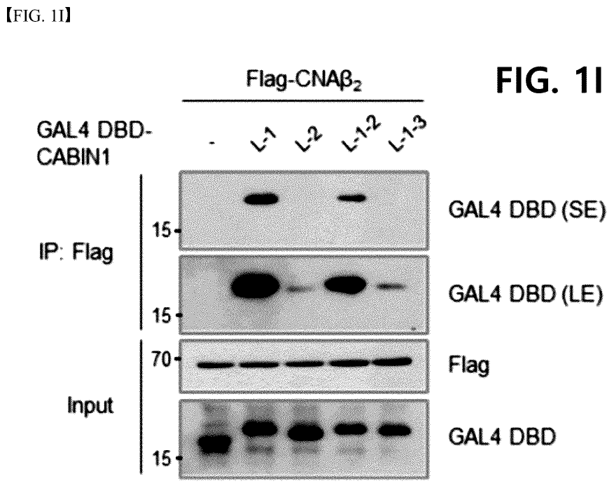

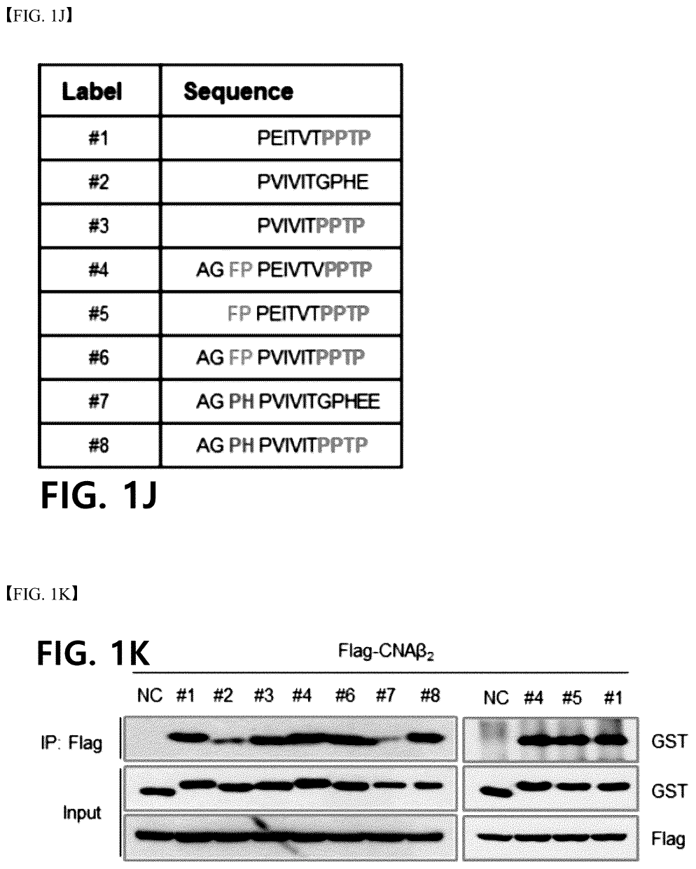

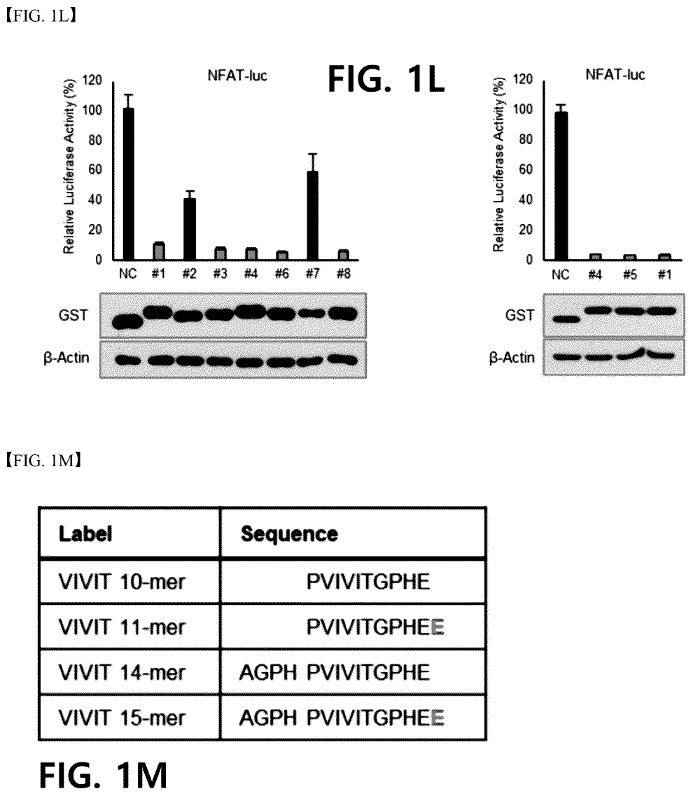

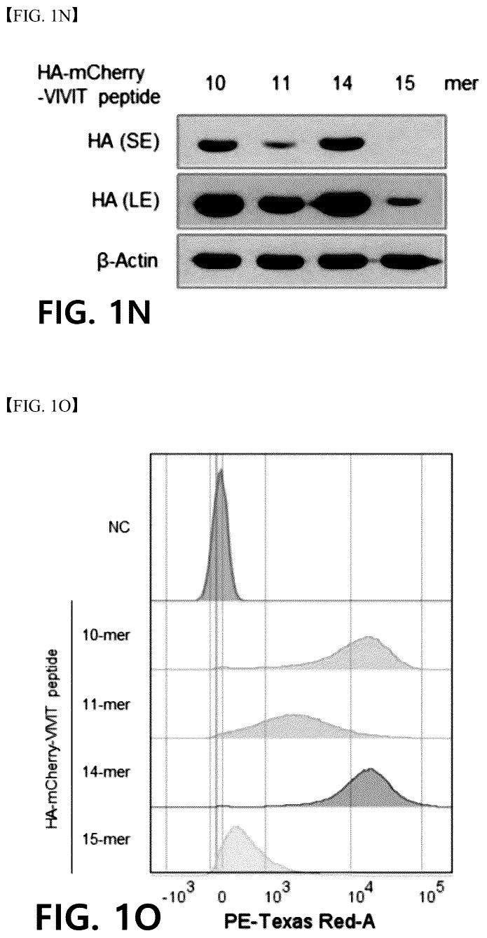

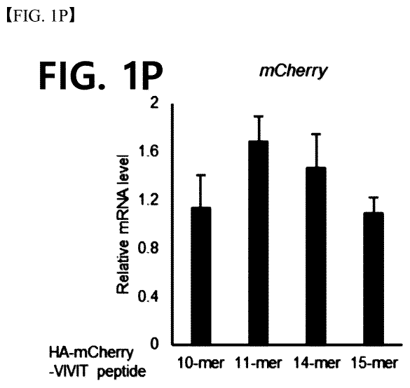

To determine the minimum binding sequence for calcineurin, the first 13-amino-acid segment (corresponding to CABIN1-L in the present invention) containing the PxIxIT motif was isolated from CABIN1-14 (FIG. 1A). Mammalian two-hybrid assay was performed using GAL4 DBD-tagged CABIN1 peptides and VP16 activation domain-tagged CNA (FIG. 1B, FIG. 1C, FIG. 1E, FIG. 1F). It was confirmed that CABIN1-14 binds to CNA, whereas the remaining sequence (CABIN1-15) does not bind. Several peptides were designed by sequentially removing CABIN1-L C-terminus amino acid residues (FIG. 1A; CABIN1-S-CABIN1-L). Mammalian two-hybrid assay showed that CABIN1-L and CABIN1-L-1 had significantly stronger affinity for CNA than CABIN1-14. However, CABIN1-L-2 completely lost its binding affinity for CNA despite its higher expression level (FIG. 1B). Thereafter, additional peptides were designed by sequentially removing CABIN1-L-1 N-terminus amino acid residues (FIG. 1A; CABIN1-L-1-1-CABIN1-L-1-4). CABIN1-L-1-1 and CABIN1-L-1-2 still bound to CNA, but CABIN1-L-1-3 lost its binding affinity (FIG. 1C). Immunoprecipitation assay using GAL4 DBD-tagged CABIN1 peptides and Flag-tagged CNA showed the same results (FIG. 1H, FIG. 1I). These results demonstrate that 10 amino acids from proline 2146 to proline 2155 of full-length CABIN1 form the minimal sequence required for interaction with calcineurin.

2.2 PPTP Sequence after PxIxIT Motif that is Important for Interaction with Calcineurin

Since proline 2155 does not belong to the PxIxIT motif, the following details were performed to clarify the role of the C-terminus “PPTP” sequence after the PxIxIT motif. To compare CABIN1 and VIVIT peptides under the same conditions, the VIVIT decamer (VIVIT 10-mer) was used as a control. A chimeric peptide was constructed by replacing the C-terminus sequences of VIVIT and CABIN1 peptides, and mammalian two-hybrid assay was performed (FIG. 1D, FIG. 1G). The C-terminus “PPTP” sequence improved the interaction with CNA by 4.76-fold (VIVIT PPTP vs. VIVIT 10-mer) and the N-terminus “PVIVIT” by 1.93-fold (VIVIT PPTP vs. L-1-2 WT). This result suggests that the C-terminus “PPTP” sequence also plays an important role in its interaction with calcineurin.

Subsequently, it was examined whether the N-terminus sequence (the “AGPH” of VIVIT peptide and the “FP” of CABIN1-L-1) before PxIxIT contributes to its interaction with calcineurin. The addition of “AG”, “PH”, or “FP” before the PxIxIT motif did not affect the interaction with calcineurin (FIG. 1J, FIG. 1K). In addition, these sequences did not affect the inhibitory effect of the calcineurin-NFAT pathway (FIG. 1L). In the case of the VIVIT peptide, it was confirmed that the C-terminus glutamate repeat reduces protein stability (FIGS. 1M to 1P).

2.3 CABIN1 Peptide as Calcineurin-NFAT Pathway Inhibitor More Potent than VIVIT Peptide

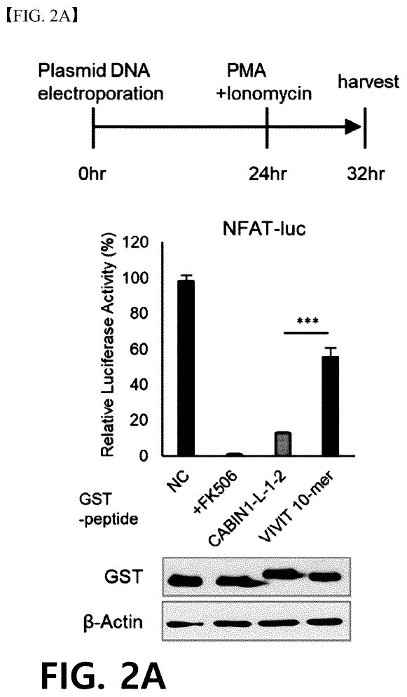

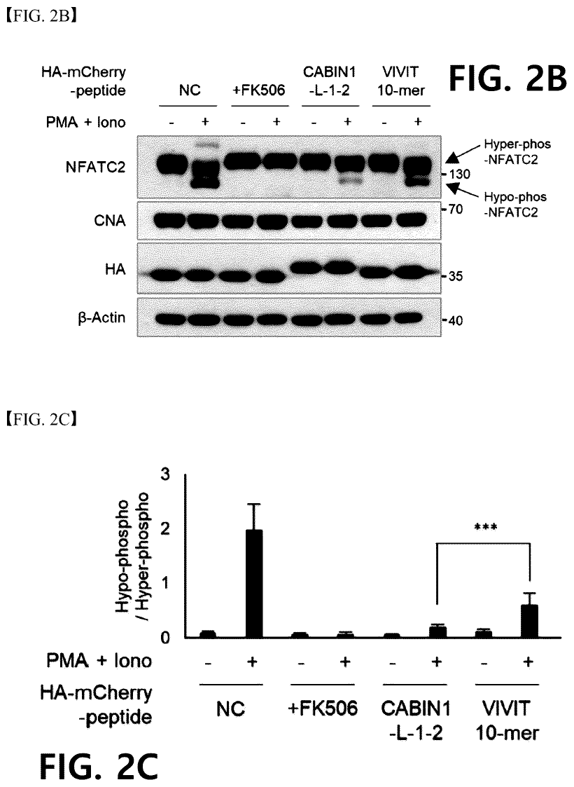

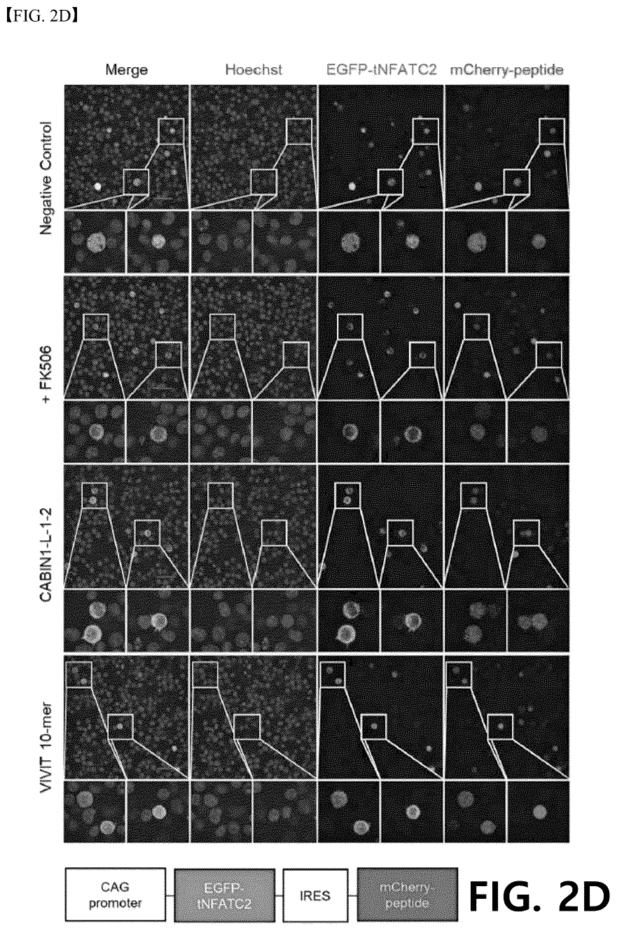

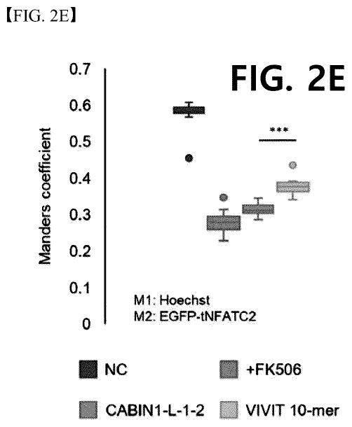

To investigate the inhibitory effect of CABIN1 peptide on the calcineurin-NFAT pathway, Jurkat T cells with a luciferase reporter gene under the control of three repeated NFAT binding sites were cotransfected with GST-tagged CABIN1-L-1-2 or VIVIT peptides. Expression of GST-tagged CABIN1-L-1-2 or VIVIT peptides upon PMA and ionomycin treatment inhibited luciferase activity compared to the negative control. The inhibitory effect of CABIN1-L-1-2 was approximately 4 times stronger than that of the VIVIT peptide (FIG. 2A). To determine whether this result was due to NFATC2 dephosphorylation by calcineurin, NFAT mobility shifting assay was performed. In T cells, NFATC1 and NFATC2 are hyperphosphorylated, and calcineurin dephosphorylates them in response to calcium signals. Hyperphosphorylated NFAT is localized in the cytoplasm, whereas dephosphorylated proteins are localized in the nucleus and move faster in the gel than hyperphosphorylated proteins. Similar to FK506, CABIN1 peptide significantly inhibited NFATC2 band shift upon PMA and ionomycin treatment (FIG. 2B). VIVIT peptide also reduced dephosphorylation of NFATC2, but its inhibitory effect was weaker than that of CABIN1 peptide (FIG. 2B, FIG. 2C).

Subsequently, to confirm the contribution of these peptides to the migration of NFAT to the nucleus, we constructed a plasmid expressing EGFP-tagged truncated NFATC2 (tNFATC2; 2-460 amino acids) and each mCherry-tagged peptide. The proportion of nuclear-localized tNFATC2 was lowest in FK506-treated cells and slightly higher in Jurkat T cells co-expressing the CABIN1-L-1-2 peptide. In addition, Jurkat T cells co-expressing VIVIT peptides had significantly higher rates of nuclear-localized tNFATC2 than cells co-expressing CABIN1-L-1-2 peptides (FIG. 2D, FIG. 2E). Overall, these results demonstrate that the CABIN1 peptide blocks dephosphorylation and nuclear translocation of NFATC2 more efficiently than the VIVIT peptide.

### 2.4 Blocking T Lymphocyte Activation by CABIN1 Peptide

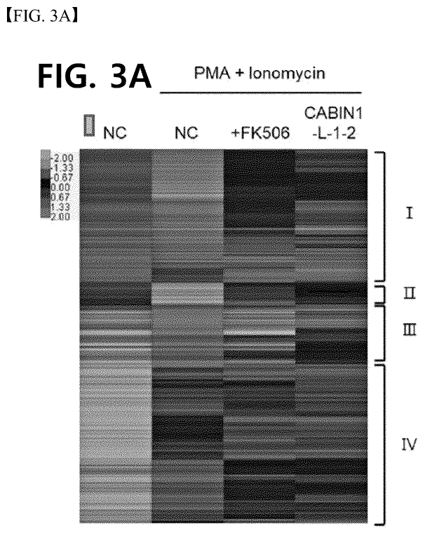

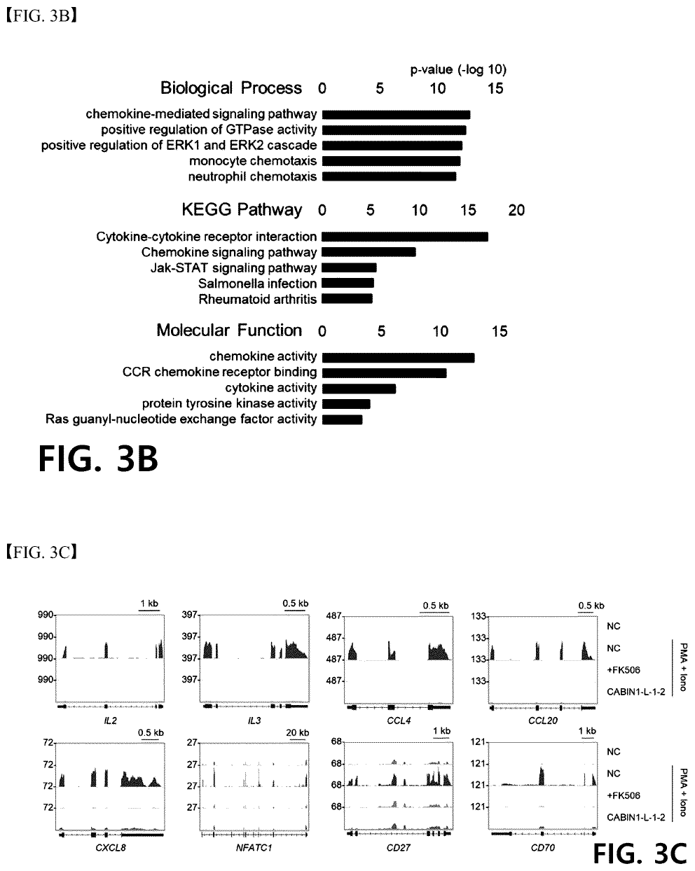

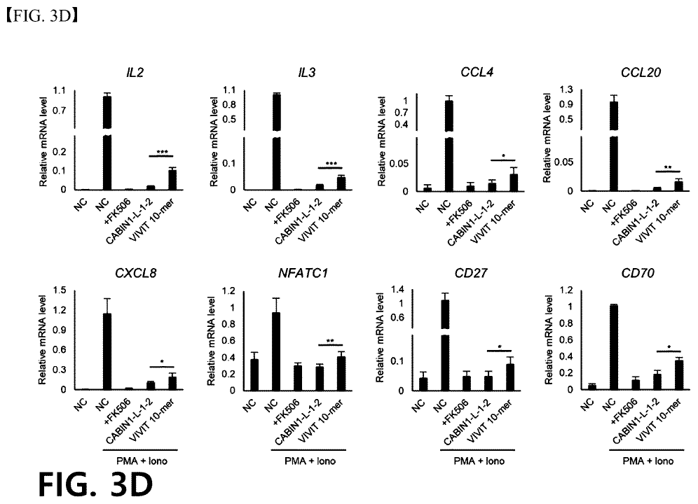

To determine whether the CABIN1 peptide blocks T lymphocyte activation by interfering with calcineurin-NFAT interaction, RNA from a negative control (mCherry) and a Jurkat T cell line stably expressing an mCherry-tagged CABIN1 peptide were sequenced. 685 genes affected by PMA and ionomycin treatment were found in negative control cells (FIG. 3A, Table 2). Of the four clusters, we focused on cluster III containing genes with increased expression levels in negative controls upon PMA and ionomycin treatment and decreased expression levels in CABIN1-expressing cells and FK506-treated cells (Table 3). Gene ontology analysis showed that the genes in cluster III are related to chemokines, cytokine signals, and immune responses (FIG. 3B). In addition, upon T-cell activation, CABIN1 peptides and FK506 reduced RNA levels of representative T-cell activation markers, including IL2, IL3, NFATC1, and various chemokine ligands (CCL4, CCL20, and CXCL8). In addition, CABIN1 peptides and FK506 inhibited CD27 and CD70, which are regulating the T-cell co-stimulation pathway (FIG. 3C). Real-time quantitative PCR analysis of these genes showed that CABIN1 peptides reduced RNA levels of these genes more effectively than VIVIT peptides (FIG. 3D).

TABLE 2

| | | NC_P + | FK506_P + | CABIN1_P + |
|---|---|---|---|---|
| | NC/ | I/ | I/ | I/ |
| NAME | FPKM | FPKM | FPKM | FPKM |
| PDGFA | 1.497 | 26.809 | 3.25 | 8.169 |
| BIN1 | 5.345 | 1.469 | 2.374 | 1.968 |
| TAP2 | 1.126 | 5.044 | 5.187 | 6.702 |
| TAL1 | 45.244 | 11.71 | 22.337 | 18.466 |
| HIVEP3 | 11.136 | 18.008 | 20.041 | 20.545 |
| ZNF827 | 0.975 | 3.401 | 1.521 | 1.372 |
| SH2D1A | 71.224 | 14.498 | 23.649 | 16.781 |
| MIR6821 | 17.553 | 62.894 | 13.626 | 25.116 |
| SLAMF7 | 0.276 | 10.24 | 3.121 | 3.544 |
| CERKL | 32.7 | 7.889 | 13.458 | 10.348 |
| METRN | 4.576 | 1.12 | 1.779 | 1.604 |
| ZNF367 | 9.126 | 2.543 | 2.354 | 2.274 |
| GKAP1 | 3.751 | 1.113 | 2.692 | 2.927 |
| LOC100506688 | 4.117 | 1.142 | 1.625 | 1.704 |
| NR4A3 | 0.024 | 4.559 | 1.927 | 1.977 |
| PXYLP1 | 12.843 | 3.772 | 5.01 | 4.92 |
| PLEC | 5.129 | 16.749 | 21.747 | 25.453 |
| SMOX | 6.97 | 27.876 | 14.9 | 18.934 |
| UBE2L6 | 30.923 | 127.161 | 164.565 | 167.347 |
| EGR2 | 0.022 | 34.883 | 11.435 | 9 |
| CAMK2D | 1.999 | 13.185 | 5.855 | 6.138 |
| APOBEC3F | 1.328 | 6.423 | 5.861 | 7.066 |
| ADORA2A | 2.492 | 11.633 | 0.998 | 1.475 |
| GTDC1 | 3.595 | 10.378 | 4.629 | 5.144 |
| SPINT1 | 3.839 | 1.14 | 1.948 | 1.712 |
| DUSP10 | 0.55 | 7.447 | 11.981 | 12.142 |
| WARS | 33.486 | 106.713 | 105.541 | 112.259 |
| ITK | 19.685 | 58.349 | 58.558 | 62.324 |
| IQGAP2 | 9.764 | 1.968 | 4.857 | 3.477 |
| ECE1 | 8.894 | 116.972 | 20.046 | 59.384 |
| ADCYAP1 | 2.988 | 0.459 | 3.646 | 3.219 |
| NFKB2 | 6.744 | 74.327 | 49.22 | 71.869 |
| IRF2 | 5.852 | 18.15 | 34.99 | 34.829 |
| TBC1D4 | 20.739 | 73.749 | 12.549 | 13.191 |
| CSF1 | 0.848 | 13.43 | 1.706 | 2.594 |
| BCL7A | 60.161 | 14.095 | 12.185 | 12.277 |
| LMNA | 0.151 | 0.699 | 4.62 | 2.786 |
| RCOR2 | 4.622 | 1.139 | 1.361 | 1.298 |
| TCF3 | 72.022 | 25.169 | 39.165 | 36.607 |
| PTPN6 | 22.163 | 77.092 | 20.906 | 20.806 |
| FAM64A | 14.202 | 3.009 | 7.456 | 6.008 |
| CHPF | 4.868 | 16.165 | 9.759 | 12.714 |
| MAZ | 235.37 | 81.339 | 112.102 | 99.474 |
| SLC22A1 | 0.158 | 61.938 | 7.005 | 42.379 |
| ZYX | 8.696 | 29.119 | 127.794 | 85.489 |
| ATPAF1 | 23.844 | 8.33 | 11.764 | 10.283 |
| IL2 | 0 | 397.807 | 0.287 | 12.964 |
| CCL3 | 0 | 52.239 | 0 | 0.502 |
| NAA16 | 8.223 | 2.868 | 3.527 | 3.877 |
| FOSL2 | 1.294 | 7.508 | 2.204 | 2.362 |
| CXCL8 | 0.037 | 26.755 | 0.937 | 3.327 |
| DBP | 6.72 | 1.783 | 2.396 | 1.773 |
| SERPINE2 | 1.408 | 5.558 | 3.936 | 3.911 |
| PHLDB2 | 6.022 | 1.831 | 3.898 | 3.404 |
| OSBPL5 | 11.263 | 1.794 | 3.354 | 3.123 |
| CNN2 | 61.031 | 5.057 | 30.77 | 18.455 |
| CDKN2D | 29.191 | 8.631 | 17.642 | 16.801 |
| PELI2 | 19.511 | 5.485 | 11.262 | 8.166 |
| LUCATI | 2.321 | 7.089 | 12.516 | 9.183 |
| CBS | 11.413 | 3.102 | 3.935 | 3.916 |
| ABTB2 | 0.787 | 5.914 | 2.549 | 6.382 |
| TAPBPL | 9.485 | 25.582 | 19.59 | 20.693 |
| CYTH1 | 56.655 | 19.659 | 33.17 | 30.15 |
| XCL1 | 0 | 49.113 | 0 | 1.144 |
| PAM | 1.154 | 5.577 | 1.483 | 1.934 |
| GLRB | 7.592 | 2.451 | 1.62 | 1.354 |
| KLF12 | 6.701 | 2.025 | 6.506 | 4.377 |
| PKM | 443.853 | 2211.937 | 1736.037 | 1838.428 |
| MAMLD1 | 0.598 | 6.963 | 5.995 | 9.949 |
| VAV1 | 34.441 | 11.911 | 30.218 | 22.182 |
| APOBEC3G | 0.737 | 22.838 | 8.803 | 9.231 |
| CFLAR | 1.799 | 3.856 | 3.438 | 3.022 |
| ZNF521 | 4.277 | 0.956 | 2.011 | 1.965 |
| PRNP | 13.132 | 43.872 | 39.685 | 41.922 |
| PPIF | 58.328 | 188.301 | 50.291 | 106.533 |
| CHPF2 | 12.181 | 33.063 | 36.42 | 34.939 |
| BEND6 | 3.986 | 1.153 | 1.416 | 1.409 |
| MAST4 | 0.876 | 5.954 | 3.498 | 5.841 |
| SORBS1 | 0.196 | 5.08 | 0.4 | 0.511 |
| IL21R | 0.1 | 34.712 | 2.244 | 5.255 |
| STAT3 | 17.67 | 52.79 | 83.385 | 74.439 |
| LTBP4 | 7.304 | 33.466 | 8.427 | 9.568 |
| PSMB10 | 31.284 | 85.264 | 104.49 | 125.268 |
| VAT1 | 100.917 | 34.64 | 44.696 | 40.336 |
| LOC653712 | 6.512 | 1.854 | 4.163 | 2.808 |
| IQCJ-SCHIP1 | 1.402 | 4.253 | 1.657 | 1.234 |
| NFATC1 | 4.511 | 15.287 | 3.793 | 3.916 |
| EHBP1L1 | 7.342 | 2.399 | 7.073 | 5.023 |
| CENPM | 44.679 | 13.062 | 13.201 | 14.415 |
| ASNS | 10.082 | 29.993 | 20.436 | 19.026 |
| LOC101927497 | 24.857 | 7.313 | 9.413 | 10.444 |
| SSBP2 | 28.793 | 4.463 | 7.127 | 5.549 |
| MAP3K7CL | 0.28 | 3.831 | 2.247 | 2.03 |
| LPAR6 | 0.354 | 1.601 | 8.449 | 4.344 |
| TRIB1 | 0.189 | 16.309 | 5.422 | 4.108 |
| HPGD | 8.191 | 1.884 | 6.064 | 3.392 |
| PPP1R16B | 2.67 | 37.059 | 29.659 | 26.306 |
| PLS3 | 0.019 | 4.717 | 0.052 | 0.37 |
| LRRC20 | 12.151 | 2.17 | 3.843 | 2.839 |
| NEDD9 | 1.089 | 4.206 | 3.748 | 4.972 |
| IL15 | 0.235 | 1.236 | 2.654 | 3.16 |
| RHOU | 3.829 | 1.044 | 1.51 | 1.378 |
| RAB37 | 3.573 | 0.196 | 2.455 | 1.369 |
| ACAP1 | 41.657 | 13.82 | 35.261 | 26.535 |
| B2M | 411.858 | 1193.352 | 1155.981 | 1302.512 |
| MYB | 89.343 | 25.529 | 58.524 | 48.107 |
| VAMP1 | 9.566 | 50.97 | 14.793 | 14.752 |
| ADAMTS17 | 5.733 | 1.475 | 0.843 | 0.882 |
| THEM6 | 11.312 | 3.511 | 3.232 | 2.542 |
| RCBTB2 | 10.697 | 1.899 | 13.008 | 7.77 |
| PKIG | 21.186 | 5.694 | 12.411 | 11.17 |
| HCAR1 | 0 | 11.451 | 7.167 | 7.943 |
| CEP70 | 34.21 | 9.737 | 14.887 | 12.745 |
| SLC35E4 | 0.887 | 9.069 | 0.952 | 1.517 |
| CD82 | 26.521 | 419.996 | 171.933 | 222.109 |
| LEF1 | 186.267 | 37.079 | 74.102 | 48.109 |
| SYK | 15.907 | 4.047 | 7.044 | 6.222 |
| ALDH1A2 | 42.14 | 4.551 | 7.041 | 6.66 |
| ITGA4 | 88.754 | 23.254 | 39.099 | 31.73 |
| SH3TC1 | 11.764 | 0.939 | 1.562 | 1.895 |
| CD274 | 0.244 | 24.463 | 7.13 | 15.92 |
| ARPP21 | 5.805 | 0.431 | 1.29 | 0.7 |
| OPTN | 1.609 | 7.963 | 12.958 | 14.333 |
| CPD | 8.281 | 35.158 | 23.208 | 26.602 |
| LDLRAD4 | 17.909 | 3.055 | 6.21 | 4.769 |
| TNFSF14 | 0.045 | 69.958 | 1.023 | 4.255 |
| KIR3DX1 | 0.01 | 0.394 | 3.048 | 3.992 |
| PDIA6 | 53.625 | 142.05 | 123.669 | 128.39 |
| HLA-A | 48.846 | 144.622 | 181.069 | 226.558 |
| TIAM2 | 0.255 | 4.095 | 0.27 | 0.301 |
| GSE1 | 39.875 | 14.157 | 17.37 | 14.556 |
| FOSL1 | 0.132 | 12.971 | 10.499 | 12.705 |
| UHRF1 | 34.265 | 7.455 | 8.288 | 6.903 |
| TMCC3 | 0.053 | 0.478 | 4.527 | 7.278 |
| ZNFX1 | 8.122 | 24.811 | 16.708 | 19.096 |
| FXYD5 | 25.643 | 101.352 | 41.346 | 42.856 |
| DUSP6 | 0.552 | 5.792 | 9.728 | 12.914 |
| IL4R | 2.023 | 39.885 | 9.842 | 12.536 |
| ACSF3 | 33.65 | 6.108 | 22.755 | 15.621 |
| CD200 | 0 | 3.759 | 0 | 0.094 |
| KIAA0247 | 4.869 | 13.963 | 8.032 | 8.83 |
| ANTXR2 | 1.504 | 20.776 | 53.159 | 55.18 |
| POU2AF1 | 1.84 | 36.298 | 2.274 | 2.971 |
| IKZF2 | 24.637 | 6.145 | 8.561 | 8.132 |
| IRF1 | 13.332 | 118.876 | 141.607 | 152.425 |
| GPR17 | 3.126 | 0.144 | 0.258 | 0.181 |
| TNFRSF10B | 7.441 | 37.653 | 25.474 | 36.431 |
| N4BP3 | 0.544 | 5.501 | 1.739 | 2.499 |
| MCM2 | 95.73 | 28.688 | 22.07 | 23.634 |
| SASH3 | 86.466 | 26.7 | 59.349 | 46.514 |
| GPC2 | 7.54 | 2.341 | 5.785 | 4.85 |
| NTRK1 | 0.167 | 7.941 | 0.836 | 1.657 |
| THEMIS | 35.003 | 7.905 | 33.406 | 35.627 |
| E2F7 | 11.171 | 2.941 | 4.531 | 4.018 |
| STMN3 | 24.035 | 5.042 | 10.423 | 7.407 |
| LGMN | 4.67 | 1.311 | 1.42 | 1.976 |
| MAFF | 1.24 | 13.462 | 16.977 | 16.464 |
| CBX2 | 7.091 | 1.05 | 2.786 | 2.454 |
| LINC01260 | 17.932 | 2.402 | 7.672 | 7.215 |
| LIMS2 | 24.38 | 4.029 | 4.122 | 4.304 |
| KLF10 | 3.03 | 16.287 | 25.627 | 15.364 |
| TIAM1 | 4.518 | 14.204 | 8.466 | 9.551 |
| ETS1 | 90.462 | 23.146 | 50.032 | 38.627 |
| MGAT4A | 17.02 | 4.173 | 8.906 | 8.882 |
| SPRY4 | 0.076 | 7.939 | 9.613 | 7.991 |
| GLRX | 33.473 | 8.559 | 25.315 | 21.013 |
| MAL | 11.053 | 0.86 | 2.664 | 2.026 |
| RASGRP2 | 16.42 | 1.08 | 3.002 | 2.335 |
| FBLN2 | 13.535 | 3.433 | 3.348 | 3.01 |
| SLC29A4 | 5.261 | 1.576 | 1.68 | 1.658 |
| TCF7 | 82.711 | 10.7 | 13.065 | 11.184 |
| ARHGAP4 | 32.153 | 10.672 | 15.97 | 16.216 |
| SCHIP1 | 1.25 | 3.933 | 1.535 | 1.333 |
| IFIT3 | 0.099 | 2.372 | 6.495 | 6.682 |
| CD1E | 45.421 | 3.154 | 4.975 | 3.521 |
| NSMF | 1.169 | 4.89 | 3.164 | 3.265 |
| QPRT | 26.609 | 7.071 | 13.402 | 10.818 |
| FAT1 | 29.711 | 10.233 | 13.036 | 11.964 |
| CCNE2 | 19.544 | 4.642 | 6.298 | 5.206 |
| NCKAP1 | 3.582 | 11.313 | 8.582 | 10.157 |
| ST3GAL6 | 1.195 | 3.863 | 4.018 | 5.239 |
| CDKN1A | 0.998 | 42.067 | 39.264 | 62.513 |
| LAIR1 | 12.869 | 1.724 | 8.298 | 7.19 |
| GXYLT2 | 13.458 | 2.813 | 5.443 | 4.24 |
| RNF212 | 3.075 | 1.394 | 1.059 | 1.401 |
| AVPR2 | 3.072 | 0.571 | 1.21 | 1.492 |
| MXD3 | 22.044 | 21.527 | 17.861 | 20.08 |
| CREB3L2 | 1.952 | 6.725 | 5.62 | 5.857 |
| PRDM8 | 1.421 | 12.326 | 39.913 | 40.783 |
| DSTYK | 2.356 | 7.198 | 3.501 | 3.595 |
| JAK2 | 2.788 | 26.566 | 5.248 | 8.45 |
| PDCD1 | 0.327 | 4.738 | 1.377 | 1.974 |
| ADA | 405.953 | 51.958 | 74.531 | 73.254 |
| ZP4 | 0 | 6.38 | 0.462 | 5.182 |
| LOC101926963 | 1.636 | 7.12 | 4.541 | 4.742 |
| FYN | 14.671 | 58.084 | 16.523 | 15.512 |
| SEPT6 | 90.554 | 29.289 | 55.091 | 44.841 |
| CCND3 | 76.176 | 19.271 | 30.402 | 25.396 |
| TAGAP | 3.351 | 128.498 | 4.352 | 8.731 |
| NT5E | 0.462 | 15.919 | 8.91 | 12.217 |
| GFOD1 | 2.283 | 12.861 | 4.187 | 6.312 |
| RASGRP1 | 27.453 | 7.034 | 4.464 | 3.909 |
| SEPT3 | 3.991 | 0.884 | 1.089 | 0.994 |
| PEX5L | 5.583 | 1.531 | 1.428 | 1.299 |
| GZMB | 0.476 | 3204.801 | 513.528 | 909.046 |
| PLAC8 | 6.697 | 1.514 | 3.158 | 1.896 |
| ZNF480 | 15.131 | 3.391 | 7.174 | 6.823 |
| MSMO1 | 17.034 | 46.91 | 59.655 | 52.664 |
| ANKEF1 | 3.896 | 0.702 | 3.081 | 2.575 |
| FADS2 | 108.804 | 34.879 | 67.903 | 48.612 |
| NFKB1 | 16.458 | 71.749 | 34.242 | 44.661 |
| LIF | 0.063 | 3.697 | 0.143 | 0.231 |
| ASB13 | 10.32 | 2.756 | 2.277 | 2.543 |
| SOX4 | 95.745 | 20.819 | 76.17 | 55.775 |
| TNFAIP3 | 4.333 | 55.781 | 25.168 | 35.424 |
| EFHD2 | 34.309 | 137.802 | 180.233 | 234.38 |
| NDRG2 | 5.775 | 0.944 | 2.881 | 2.149 |
| COL27A1 | 4.149 | 17.216 | 4.463 | 5.729 |
| IL27RA | 2.341 | 9.341 | 10.817 | 10.764 |
| ZFP36L1 | 5.72 | 143.376 | 153.648 | 136.279 |
| PHACTR2 | 1.659 | 9.467 | 2.927 | 2.791 |
| POU2F2 | 0.87 | 12.177 | 3.317 | 4.109 |
| EMP1 | 0.156 | 8.12 | 4.613 | 4.589 |
| ATP6V0A4 | 0.042 | 2.687 | 21.385 | 18.558 |
| POTEE | 3.342 | 1.022 | 1.42 | 1.196 |
| APH1B | 5.907 | 1.729 | 5.12 | 4.206 |
| SPRY1 | 2.079 | 12.391 | 4.074 | 4.901 |
| MT2A | 71.941 | 413.047 | 369.658 | 409.501 |
| CARHSP1 | 39.677 | 11.699 | 20.082 | 14.562 |
| AFF2 | 7.635 | 1.272 | 4.374 | 3.175 |
| SERINC5 | 13.434 | 3.2 | 3.184 | 3.312 |
| PDGFC | 17.949 | 2.362 | 2.292 | 2.913 |
| BCL2L1 | 31.375 | 113.956 | 39.402 | 41.852 |
| STX1A | 1.467 | 10.488 | 16.1 | 13.882 |
| SBK1 | 9.277 | 2.256 | 9.239 | 5.945 |
| IL10RA | 0.471 | 1.592 | 2.828 | 3.208 |
| SERPINB8 | 8.633 | 26.053 | 23.788 | 23.508 |
| IL15RA | 0.203 | 3.751 | 5.979 | 7.481 |
| TIAF1 | 15.536 | 5.204 | 12.479 | 9.375 |
| SEPT9 | 97.462 | 23.985 | 55.265 | 39.741 |
| PTPN7 | 43.729 | 160.438 | 93.59 | 78.61 |
| XCL2 | 0.056 | 70.747 | 0 | 2.074 |
| MIR3652 | 96.162 | 486.958 | 547.182 | 567.901 |
| KREMEN1 | 6.452 | 2.248 | 3.122 | 2.958 |
| NDRG1 | 5.346 | 28.817 | 126.361 | 128.386 |
| MICAL2 | 0.865 | 3.866 | 1.763 | 1.84 |
| TBKBP1 | 2.812 | 38.348 | 16.894 | 22.205 |
| KCNK1 | 10.828 | 36.893 | 10.967 | 13.139 |
| SERTAD1 | 3.825 | 17.874 | 16.162 | 14.426 |
| SH2D2A | 2.277 | 41.819 | 20.658 | 22.586 |
| ODC1 | 133.175 | 391.769 | 245.466 | 374.309 |
| SIVA1 | 172.026 | 51.28 | 50.269 | 51.964 |
| BAHCC1 | 9.923 | 0.482 | 1 | 0.873 |
| APOL1 | 0.065 | 1.03 | 4.036 | 4.498 |
| FKBP5 | 24.757 | 8.891 | 8.687 | 7.791 |
| DCBLD2 | 2.644 | 12.227 | 13.786 | 15.588 |
| FLI1 | 19.369 | 6.572 | 13.241 | 10.752 |
| DNAJB5 | 3.056 | 16.902 | 19.238 | 19.35 |
| CREB5 | 0.031 | 4.533 | 0.577 | 1.966 |
| APOBEC3B-AS1 | 0.045 | 16.621 | 13.251 | 20.386 |
| SLAMF1 | 0.042 | 5.663 | 0.217 | 0.275 |
| C11orf74 | 10.295 | 0.715 | 0.365 | 0.392 |
| SMAD1 | 7.505 | 2.417 | 4.873 | 3.995 |
| NSG1 | 11.29 | 2.308 | 4.302 | 3.929 |
| DTX1 | 18.177 | 2.218 | 4.414 | 3.245 |
| GFRA1 | 3.744 | 0.902 | 0.991 | 0.76 |
| CECR1 | 6.961 | 1.828 | 5.304 | 3.984 |
| ALOX5AP | 22.944 | 7.373 | 92.971 | 39.735 |
| XRCC6BP1 | 20.482 | 6.203 | 9.744 | 9.204 |
| NOTCH3 | 6.853 | 0.53 | 0.902 | 0.859 |
| RUNX3 | 4.704 | 25.871 | 23.624 | 28.166 |
| LEF1-AS1 | 2.965 | 1.506 | 3.747 | 2.388 |
| MIR6775 | 12.409 | 50.527 | 19.643 | 21.57 |
| RNF19A | 6.138 | 25.66 | 12.334 | 12.101 |
| C10orf55 | 0.1 | 11.26 | 7.632 | 15.779 |
| STAT2 | 7.544 | 21.144 | 37.805 | 36.117 |
| ANO8 | 4.062 | 0.957 | 3.26 | 2.721 |
| STAU2 | 11.237 | 4.171 | 7.124 | 5.719 |
| NME4 | 42.216 | 14.381 | 16.395 | 17.501 |
| PCNA-AS1 | 206.038 | 72.084 | 70.937 | 71.806 |
| BCL11B | 17.763 | 3.329 | 3.688 | 3.353 |
| ATP2A3 | 113.023 | 15.616 | 47.81 | 36.996 |
| ARHGEF3 | 6.146 | 31.337 | 8.637 | 8.674 |
| CHST12 | 23.741 | 8.104 | 13.527 | 12.639 |
| NLRC3 | 7.329 | 2.409 | 7.789 | 5.507 |
| SPSB1 | 0.944 | 4.094 | 10.137 | 9.593 |
| RRAS2 | 5.694 | 15.372 | 18.257 | 20.023 |
| LRRC8C | 2.556 | 31.63 | 10.058 | 11.753 |
| RUNX1 | 73.877 | 25.016 | 37.963 | 28.867 |
| YPEL1 | 7.804 | 2.488 | 5.628 | 4.38 |
| SMPD1 | 1.941 | 7.046 | 20.599 | 18.206 |
| CSF2 | 0 | 23.497 | 0 | 0.314 |
| TPM4 | 175.429 | 60.961 | 202.311 | 178.336 |
| ITGAL | 17.143 | 2.42 | 10.212 | 7.674 |
| SLC35G2 | 3.937 | 12.866 | 10.118 | 11.385 |
| ATHL1 | 11.436 | 2.861 | 4.597 | 6.014 |
| BCL6 | 1.073 | 9.143 | 15.181 | 15.55 |
| DTL | 26.496 | 5.74 | 5.862 | 5.275 |
| SGK1 | 0.854 | 22.157 | 76.683 | 105.258 |
| TPST2 | 10.644 | 42.955 | 21.077 | 29.147 |
| HLA-B | 14.546 | 137.409 | 116.079 | 160.359 |
| XIRP1 | 0 | 63.877 | 6.348 | 13.281 |
| SRGN | 4.303 | 14.597 | 10.812 | 9.743 |
| NREP | 33.624 | 8.235 | 9.925 | 6.879 |
| CHEK2 | 23.817 | 6.012 | 8.907 | 7.751 |
| TCF12 | 80.871 | 23.735 | 34.798 | 29.903 |
| GNG4 | 2.073 | 10.63 | 5.365 | 7.415 |
| SLC27A2 | 4.292 | 13.006 | 41.996 | 41.908 |
| AGK | 14.395 | 39.056 | 14.029 | 14.306 |
| GAD1 | 0.317 | 3.943 | 2.246 | 2.238 |
| NRP1 | 0.177 | 3.835 | 14.572 | 14.016 |
| TRIM21 | 9.766 | 26.937 | 45.856 | 42.08 |
| KIAA0101 | 43.447 | 11.336 | 21.727 | 16.766 |
| CD97 | 10.97 | 49.481 | 19.394 | 21.496 |
| TLE1 | 5.785 | 1.657 | 1.864 | 1.647 |
| PPP1R15A | 10.908 | 54.597 | 77.478 | 76.265 |
| IRF4 | 0.034 | 8.439 | 0.423 | 2.092 |
| RTN4R | 5.402 | 0.969 | 2.449 | 1.548 |
| NPDC1 | 3.008 | 8.759 | 4.328 | 5.64 |
| ZNF280D | 5.448 | 1.494 | 2.345 | 2.132 |
| IRF7 | 4.722 | 14.286 | 21.167 | 21.372 |
| CBFA2T3 | 27.65 | 5.839 | 12.236 | 11.821 |
| MAGEA4 | 1.603 | 5.263 | 3.908 | 3.775 |
| CCL20 | 0 | 59.393 | 0 | 1.279 |
| TNFSF10 | 1.024 | 9.324 | 24.883 | 49.452 |
| EGR1 | 2.036 | 64.209 | 218.444 | 123.294 |
| CABP1 | 1.702 | 5.834 | 1.201 | 1.689 |
| RORA | 4.213 | 1.245 | 2.502 | 1.998 |
| BCL2A1 | 0.067 | 222.759 | 21.118 | 51.928 |
| NCOA3 | 3.871 | 14.211 | 9.949 | 11.775 |
| MFAP4 | 3.474 | 0.577 | 0.663 | 0.663 |
| LMO1 | 4.098 | 1.424 | 1.094 | 0.954 |
| TGFBR2 | 6.333 | 43.071 | 36.615 | 36.254 |
| SCG2 | 1.46 | 54.619 | 159.394 | 86.711 |
| MZB1 | 101.866 | 23.221 | 22.015 | 23.597 |
| TTLL1 | 7.109 | 2.235 | 3.69 | 3.295 |
| CHRNA3 | 6.949 | 2.269 | 4.293 | 3.499 |
| ZNF704 | 0.107 | 4.985 | 0.014 | 0.183 |
| MCTP2 | 0.583 | 3.508 | 5.326 | 3.187 |
| BCL3 | 0.067 | 1.132 | 14.57 | 17.15 |
| CD70 | 1.101 | 61.146 | 3.005 | 7.606 |
| ARHGAP25 | 2.976 | 0.812 | 3.447 | 2.157 |
| RB1 | 71.078 | 24.713 | 62.321 | 49.538 |
| PI16 | 24.532 | 2.217 | 17.07 | 9.625 |
| TDRD9 | 1.428 | 25.902 | 1.245 | 1.888 |
| CCDC15 | 6.226 | 2.079 | 3.535 | 2.926 |
| PDLIM1 | 21.843 | 3.522 | 6.874 | 5.135 |
| ZFP36L2 | 30.948 | 8.275 | 13.343 | 9.774 |
| CD1B | 25.556 | 4.688 | 4.115 | 4.733 |
| ARHGAP31 | 1.341 | 8.14 | 10.889 | 11.177 |
| TBC1D5 | 9.718 | 1.789 | 4.088 | 3.021 |
| BIRC3 | 2.957 | 70.496 | 30.767 | 58.303 |
| CBLB | 3.959 | 11.37 | 8.881 | 7.757 |
| IL21R-AS1 | 0.048 | 23.616 | 1.927 | 3.991 |
| LINC00659 | 0.604 | 3.527 | 10.239 | 9.828 |
| TRAF3IP3 | 25.055 | 4.597 | 15.012 | 11.237 |
| SLAMF8 | 0.199 | 3.509 | 5.282 | 5.828 |
| WDR76 | 23.051 | 8.198 | 8.368 | 8.215 |
| GPR68 | 0.558 | 2.704 | 8.916 | 11.224 |
| CXCR4 | 188.803 | 26.517 | 38.979 | 34.014 |
| MIR22HG | 1.343 | 6.088 | 9.248 | 8.63 |
| DENND5A | 8.353 | 30.108 | 24.792 | 37.896 |
| ATP6V1B2 | 33.172 | 129.395 | 50.672 | 60.661 |
| ZNF652 | 3.976 | 1.292 | 2.631 | 2.469 |
| SPTBN2 | 4.048 | 0.792 | 2.135 | 2.053 |
| RGS2 | 0.977 | 9.667 | 3.365 | 3.39 |
| NOTCH2 | 3.645 | 11.923 | 10.308 | 12.916 |
| CXCL11 | 0 | 0.648 | 0.695 | 3.793 |
| C21orf58 | 12.045 | 3.947 | 5.847 | 6.073 |
| CCL3L3 | 0 | 6.524 | 0 | 0.079 |
| GDPD5 | 4.329 | 12.625 | 29.626 | 39.139 |
| VANGL1 | 21.457 | 5.713 | 9.553 | 8.842 |
| TM4SF1 | 0 | 3.268 | 0.439 | 0.907 |
| PARP9 | 6.919 | 50.016 | 84.306 | 84.386 |
| CTTN | 0.428 | 16.075 | 11.037 | 15.629 |
| VGF | 0.282 | 11.869 | 59.749 | 54.294 |
| CCDC71L | 2.517 | 15.309 | 13.763 | 16.319 |
| RGS16 | 0.077 | 29.102 | 1.172 | 5.595 |
| LINC00426 | 3.591 | 0.365 | 1.041 | 0.675 |
| IL3 | 0 | 229.054 | 0.062 | 6.298 |
| CFD | 3.255 | 0.818 | 0.937 | 1.22 |
| PXMP2 | 18.061 | 5.569 | 5.624 | 7.334 |
| LAX1 | 8.344 | 24 | 8.052 | 7.578 |
| MCM4 | 89.017 | 30.965 | 26.368 | 27.291 |
| SLC25A45 | 7.535 | 1.819 | 3.26 | 2.626 |
| DOK2 | 8.332 | 36.459 | 27.372 | 27.542 |
| JUNB | 12.877 | 53.734 | 55.036 | 56.126 |
| MYO7B | 59.265 | 8.343 | 15.796 | 12.661 |
| DUSP2 | 18.632 | 472.111 | 123.613 | 97.06 |
| IGLL1 | 34.29 | 2.82 | 3.429 | 3.244 |
| C1orf233 | 4.195 | 0.525 | 0.79 | 0.674 |
| TBC1D10C | 17.466 | 4.364 | 23.871 | 15.764 |
| CYB5RL | 3.61 | 1.139 | 1.807 | 1.449 |
| DPYSL2 | 15.045 | 5.427 | 11 | 9.177 |
| GLIPR2 | 16.149 | 5.189 | 15.84 | 11.773 |
| RGS3 | 0.249 | 5.068 | 2.142 | 2.303 |
| BHLHE40 | 2.499 | 6.901 | 6.563 | 9.368 |
| PPFIBP1 | 4.095 | 10.885 | 12.205 | 12.117 |
| GNAQ | 43.005 | 11.031 | 21.057 | 17.078 |
| XYLB | 8.978 | 2.856 | 1.506 | 2.311 |
| EPAS1 | 0.188 | 0.797 | 5.846 | 4.197 |
| NKX3-1 | 23.419 | 6.852 | 7.464 | 8.412 |
| SERPINB2 | 0.28 | 85.882 | 2.905 | 14.103 |
| ADD3 | 21.964 | 4.318 | 13.6 | 9.536 |
| SH2B3 | 7.761 | 23.644 | 13.899 | 14.803 |
| CCDC64 | 5.477 | 33.545 | 10.044 | 11.039 |
| C9orf9 | 12.861 | 3.075 | 7.304 | 6.009 |
| FST | 0 | 15.319 | 2.244 | 6.872 |
| RORB | 3.524 | 0.72 | 3.782 | 3.292 |
| OSBPL3 | 8.455 | 22.99 | 24.517 | 23.298 |
| XBP1 | 17.617 | 54.64 | 43.828 | 48.525 |
| SLC2A3 | 2.444 | 19.284 | 45.525 | 40.147 |
| ARHGEF6 | 55.593 | 12.62 | 35.366 | 27.254 |
| LRRC8B | 2.89 | 28.699 | 3.555 | 4.298 |
| ENHO | 3.463 | 0.34 | 1.746 | 1.223 |
| KIF3B | 7.124 | 25.597 | 13.87 | 14.583 |
| STAT5A | 1.417 | 9.105 | 3.347 | 3.055 |
| CD1A | 39.249 | 3.977 | 4.589 | 4.51 |
| TNRC6C | 10.066 | 3.479 | 4.711 | 4.485 |
| AQP3 | 36.184 | 11.327 | 66.037 | 52.98 |
| SHC4 | 0.069 | 4.391 | 0.663 | 0.686 |
| TMTC4 | 7.082 | 1.794 | 2.677 | 3.005 |
| DNPEP | 17.801 | 50.94 | 31.154 | 43.482 |
| MRPL39 | 38.485 | 238.099 | 132.452 | 188.834 |
| PLAU | 0.66 | 61.729 | 45.171 | 82.517 |
| NEXN | 0.226 | 2.551 | 3.661 | 5.587 |
| IL4I1 | 5.799 | 6.34 | 7.078 | 7.279 |
| HAPLN3 | 1.442 | 48.075 | 48.4 | 58.345 |
| RAP1GAP | 6.799 | 2.193 | 1.878 | 1.799 |
| CXCL3 | 0.245 | 4.241 | 3.147 | 7.007 |
| TUBB3 | 11.952 | 2.205 | 9.713 | 5.423 |
| C16orf46 | 1.017 | 3.001 | 2.655 | 3.705 |
| PMEPA1 | 34.688 | 2.175 | 3.548 | 2.393 |
| TSHR | 7.258 | 0.256 | 1.535 | 1.574 |
| FAM46C | 6.988 | 2.032 | 5.191 | 5.693 |
| CD1D | 17.887 | 3.263 | 2.553 | 3.093 |
| PTK2 | 5.74 | 20.624 | 13.215 | 13.391 |
| ASAP1 | 4.678 | 18.37 | 7.765 | 9.052 |
| PTPN22 | 8.928 | 45.38 | 6.37 | 5.119 |
| IER2 | 19.131 | 59.903 | 181.539 | 126.884 |
| CD83 | 14.024 | 46.169 | 35.907 | 41.173 |
| TNFRSF4 | 2.597 | 31.683 | 2.933 | 11.916 |
| NRROS | 57.345 | 20.402 | 36.775 | 26.059 |
| LYPD3 | 1.545 | 4.832 | 2.727 | 2.662 |
| OAS3 | 4.289 | 11.893 | 13.085 | 18.24 |
| LRRC45 | 14.257 | 3.147 | 4.142 | 4.264 |
| DHFR | 20.499 | 7.117 | 7.656 | 6.668 |
| NDFIP1 | 19.638 | 54.615 | 22.485 | 24.432 |
| IFFO2 | 1.647 | 5.162 | 2.752 | 3.163 |
| IL16 | 1.427 | 6.75 | 10.552 | 8.304 |
| TRAF1 | 0.528 | 46.848 | 11.176 | 24.962 |
| HSP90B1 | 148.345 | 846.287 | 877.39 | 910.415 |
| CNR2 | 4.165 | 0.223 | 0.674 | 0.552 |
| SIX6 | 52.35 | 13.291 | 15.688 | 19.444 |
| IMPA2 | 11.686 | 3.007 | 3.015 | 2.882 |
| IGSF9B | 2.045 | 8.871 | 7.175 | 8.992 |
| PWWP2B | 4.987 | 1.248 | 2.769 | 3.446 |
| GPR108 | 17.207 | 47.945 | 31.682 | 37.394 |
| CORO2A | 5.418 | 1.42 | 1.862 | 1.623 |
| XAF1 | 1.876 | 8.299 | 15.924 | 16.914 |
| NEIL3 | 26.1 | 5.801 | 9.418 | 8.336 |
| CXCL9 | 0 | 1.581 | 0.839 | 7.221 |
| NR4A1 | 2.716 | 25.797 | 52.553 | 34.85 |
| LAMP3 | 8.064 | 31.61 | 6.412 | 9.828 |
| GAMT | 27.381 | 9.875 | 10.915 | 11.98 |
| TRIM22 | 0.102 | 0.842 | 2.908 | 3.316 |
| TSPAN2 | 4.997 | 0.893 | 2.414 | 2.119 |
| APOL2 | 11.71 | 63.794 | 89.267 | 102.003 |
| LOC729603 | 1.468 | 5.071 | 2.179 | 3.329 |
| HDC | 0.024 | 14.173 | 26.3 | 23.209 |
| SECISBP2L | 6.651 | 19.048 | 13.57 | 12.883 |
| PKIA | 6.444 | 49.019 | 13.962 | 18.138 |
| CD55 | 3.587 | 39.451 | 6.015 | 12.082 |
| PHEX | 0.188 | 17.377 | 0.213 | 0.786 |
| GRK5 | 5.451 | 21.464 | 8.481 | 8.975 |
| PLEKHO1 | 55.893 | 18.221 | 47.519 | 36.376 |
| FMNL2 | 2.395 | 6.813 | 2.726 | 3.019 |
| TTC28 | 4.28 | 1.23 | 2.798 | 1.926 |
| CCL4L2 | 0 | 12.758 | 0.087 | 0.328 |
| CXCL10 | 0 | 1.106 | 0.352 | 6.288 |
| CSRNP1 | 2.387 | 26.898 | 26.052 | 23.093 |
| PHF7 | 6.667 | 2.026 | 3.968 | 3.075 |
| ATXN1 | 0.733 | 3.381 | 1.798 | 2.083 |
| CHRNA9 | 4.187 | 1.099 | 4.046 | 2.572 |
| LINC00528 | 6.661 | 1.762 | 5.759 | 5.927 |
| GRAP2 | 30.381 | 10.47 | 19.094 | 11.197 |
| CRIM1 | 0.268 | 3.029 | 0.305 | 0.333 |
| FAM111B | 13.399 | 3.849 | 5.291 | 4.22 |
| LAIR2 | 0 | 1.933 | 2.203 | 3.695 |
| GZMH | 0.283 | 45.057 | 7.694 | 13.893 |
| GNG2 | 26.471 | 8.896 | 25.67 | 17.754 |
| ARHGAP23 | 4.013 | 1.003 | 2.576 | 1.985 |
| TNFRSF14 | 1.345 | 4.446 | 12.35 | 12.09 |
| E2F2 | 13.902 | 2.827 | 3.317 | 3.419 |
| SPRY2 | 4.541 | 18.913 | 16.882 | 17.876 |
| LDLRAP1 | 3.217 | 0.374 | 0.778 | 0.878 |
| SEMA7A | 3.218 | 42.26 | 26.567 | 34.623 |
| HHIP | 18.16 | 3.865 | 9.345 | 6.793 |
| FASLG | 0.034 | 7.22 | 1.518 | 1.715 |
| HIVEP2 | 2.983 | 22.441 | 7.494 | 9.184 |
| SYTL1 | 14.703 | 4.911 | 15.004 | 12.555 |
| SH3RF2 | 0.546 | 8.116 | 27.941 | 32.15 |
| PODXL | 4.505 | 0.916 | 1.86 | 1.509 |
| SREBF1 | 27.932 | 7.792 | 14.076 | 12.747 |
| DFNA5 | 2.024 | 13.031 | 12.016 | 9.395 |
| TIMP2 | 9.09 | 1.3 | 1.948 | 1.952 |
| MIR6891 | 3.784 | 30.185 | 24.428 | 31.815 |
| KIAA0922 | 40.863 | 14.062 | 12.92 | 12.911 |
| GEM | 0.129 | 7.322 | 2.495 | 2.963 |
| AGO2 | 7.51 | 21.395 | 13.242 | 14.51 |
| SH2D5 | 0.508 | 1.613 | 1.469 | 3.557 |
| APOBEC3B | 0.164 | 46.046 | 41.16 | 59.895 |
| ISM1 | 0.798 | 56.893 | 0.726 | 3.737 |
| UHRF1BP1L | 4.289 | 13.987 | 7.474 | 7.889 |
| SOCS1 | 0.361 | 5.77 | 9.708 | 11.547 |
| ADAM19 | 0.115 | 3.123 | 5.532 | 4.257 |
| ERO1LB | 1.452 | 4.009 | 5.101 | 4.345 |
| ICAM1 | 1.13 | 8.163 | 12.704 | 12.914 |
| ADORA2B | 2.309 | 10.507 | 8.219 | 6.969 |
| SOCS3 | 0.412 | 6.909 | 8.922 | 9.392 |
| KIF1B | 3.377 | 17.954 | 11.727 | 13.254 |
| FBXO34 | 11.735 | 48.015 | 14.612 | 18.386 |
| IRF9 | 4.818 | 29.184 | 39.109 | 39.375 |
| SOSTDC1 | 11.831 | 1.679 | 8.472 | 7.34 |
| IFNG | 0 | 28.865 | 3.8 | 8.858 |
| LINC00152 | 3.473 | 60.002 | 32.822 | 31.801 |
| E2F1 | 30.985 | 10.056 | 7.783 | 8.062 |
| NDFIP2 | 3.082 | 14.277 | 13.13 | 12.598 |
| MAP2K3 | 21.676 | 144.057 | 41.815 | 43.237 |
| KCTD11 | 4.27 | 1.086 | 3.399 | 2.303 |
| USP30-AS1 | 0.48 | 3.084 | 6.464 | 9.017 |
| ARC | 0.021 | 22.76 | 9.966 | 10.186 |
| FYB | 22.142 | 5.913 | 11.107 | 10.258 |
| PRKCA | 40.602 | 9.875 | 14.104 | 11.132 |
| APOBEC3C | 3.797 | 16.567 | 16.851 | 14.323 |
| BACH2 | 2.387 | 57.828 | 29.7 | 36.118 |
| TRAT1 | 13.116 | 1.838 | 7.968 | 5.507 |
| RMI1 | 18.909 | 6.379 | 8.984 | 8.198 |
| GBP3 | 2.313 | 34.082 | 69.945 | 68.757 |
| TBC1D20 | 11.844 | 35.808 | 17.7 | 18.427 |
| HHIP-AS1 | 66.764 | 14.584 | 32.468 | 29.854 |
| CDCA7 | 92.001 | 24.768 | 26.911 | 21.851 |
| PDE3B | 14.442 | 4.977 | 9.108 | 5.194 |
| ARAP2 | 2.197 | 10.711 | 15.321 | 13.458 |
| C1QL1 | 3.175 | 0.772 | 1.444 | 0.756 |
| CCL1 | 0 | 9.86 | 0 | 0.415 |
| PIM3 | 37.434 | 180.382 | 42.827 | 72.895 |
| CBX6 | 19.627 | 62.434 | 42.006 | 65.604 |
| RAG2 | 10.016 | 0.244 | 0.173 | 0.385 |
| RDH10 | 4.125 | 19.889 | 5.856 | 5.524 |
| SLC2A4RG | 5.881 | 1.461 | 2.367 | 2.168 |
| GBP5 | 0.062 | 130.751 | 197.514 | 262.945 |
| ST8SIA4 | 3.394 | 9.53 | 11.077 | 8.921 |
| MBOAT2 | 5.378 | 1.806 | 3.773 | 3.202 |
| CHI3L2 | 160.778 | 54.252 | 58.214 | 51.627 |
| SERPINE1 | 0 | 2.076 | 6.785 | 4.431 |
| SIT1 | 51.291 | 7.298 | 48.56 | 34.287 |
| GPR183 | 0.281 | 1.519 | 5.776 | 4.735 |
| PHLDA1 | 0.64 | 30.9 | 65.703 | 52.893 |
| GPR3 | 1.185 | 10.707 | 7.96 | 9.964 |
| MMP10 | 0 | 188.369 | 11.973 | 22.372 |
| SLC7A5 | 104.518 | 500.415 | 207.842 | 226.44 |
| MYO1G | 64.829 | 11.602 | 98.59 | 65.452 |
| APOL6 | 2.603 | 27.312 | 46.937 | 46.762 |
| FURIN | 7.171 | 19.805 | 23.264 | 29.008 |
| CRTAM | 0.104 | 100.175 | 3.325 | 9.51 |
| PARP14 | 7.365 | 40.426 | 65.432 | 72.911 |
| ID2 | 2.484 | 27.855 | 13.425 | 15.335 |
| ENC1 | 2.218 | 14.108 | 9.299 | 9.268 |
| NFKBID | 3.606 | 10.726 | 6.668 | 8.294 |
| PELI1 | 5.755 | 16.085 | 8.175 | 8.465 |
| LINC00539 | 11.913 | 1.403 | 5.004 | 2.351 |
| NFKBIA | 9.923 | 79.3 | 39.193 | 70.837 |
| CCND2 | 0.321 | 6.436 | 0.558 | 0.99 |
| IL2RA | 0.01 | 16.919 | 5.833 | 10.904 |
| SH3BGRL3 | 111.636 | 355.335 | 408.163 | 351.357 |
| SELPLG | 21.352 | 1.378 | 5.062 | 3.724 |
| CXCR3 | 0.604 | 14.322 | 0.503 | 1.803 |
| NDST3 | 10.786 | 1.405 | 10.369 | 5.621 |
| PIDD1 | 11.655 | 3.683 | 5.28 | 4.471 |
| GINS2 | 56.457 | 18.741 | 16.935 | 16.516 |
| IRF8 | 1.168 | 14.813 | 1.19 | 1.914 |
| NFE2L3 | 8.648 | 31.259 | 15.944 | 28.495 |
| HSP90B2P | 2.295 | 9.218 | 8.45 | 9.619 |
| IFIH1 | 2.155 | 8.429 | 11.544 | 12.37 |
| REL | 2.654 | 20.219 | 4.749 | 5.699 |
| SAMD1 | 30.407 | 9.053 | 15.561 | 12.809 |
| EVI2A | 1.069 | 11.844 | 15.078 | 10.047 |
| TBX21 | 0.171 | 57.479 | 24.784 | 39.746 |
| PCBP3 | 0.923 | 3.111 | 4.527 | 4.642 |
| CCL4 | 0 | 247.287 | 0 | 4.062 |
| SMIM14 | 4.316 | 1.197 | 7.677 | 3.572 |
| KCNK5 | 3.84 | 14.865 | 3.906 | 4.904 |
| ST3GAL6-AS1 | 1.309 | 9.671 | 9.039 | 8.479 |
| TMEM229B | 1.854 | 7.079 | 6.813 | 7.747 |
| ACTG1 | 1354.665 | 452.78 | 1002.221 | 799.981 |
| JAK3 | 1.607 | 6.336 | 8.03 | 8.871 |
| THY1 | 11.123 | 2.912 | 3.994 | 3.297 |
| ATP2B4 | 18.152 | 1.829 | 16.449 | 10.46 |
| STAT1 | 15.878 | 317.544 | 611.139 | 527.58 |
| GBP1 | 0.504 | 27.863 | 70.024 | 87.487 |
| RELB | 1.091 | 50.179 | 29.337 | 40.689 |
| PTPRE | 5.085 | 14.58 | 14.031 | 14.28 |
| GBP4 | 0 | 2.965 | 4.384 | 9.209 |
| MAP2K6 | 6.52 | 1.459 | 1.149 | 0.955 |
| CD69 | 2.921 | 416.388 | 125.597 | 121.846 |
| PMAIP1 | 10.951 | 37.079 | 26.095 | 37.469 |
| CD1C | 8.708 | 1.918 | 3.288 | 2.59 |
| TSPAN7 | 145.387 | 35.603 | 44.718 | 42.604 |
| ENPP1 | 0.211 | 9.218 | 124.248 | 51.27 |
| GBP2 | 0.015 | 45.346 | 27.591 | 38.881 |
| METRNL | 0.141 | 6.604 | 7.501 | 7.881 |
| NXN | 12.588 | 1.19 | 2.574 | 1.571 |
| DUSP5 | 0.991 | 95.69 | 116.404 | 157.077 |
| PLK3 | 4.432 | 14.807 | 5.257 | 7.036 |
| CTDSPL | 3.213 | 1.033 | 1.758 | 1.836 |
| NPY | 0 | 4.94 | 4.771 | 4.799 |
| TNFRSF12A | 0.792 | 3.349 | 8.559 | 6.523 |
| DNAJC1 | 5.462 | 19.836 | 13.765 | 13.844 |
| TM7SF3 | 62.693 | 19.435 | 29.483 | 25.052 |
| TNS3 | 0.008 | 13.196 | 0.678 | 1.418 |
| STX11 | 0.534 | 5.108 | 2.733 | 2.972 |
| TUBB2A | 4.011 | 18.285 | 7.594 | 10.912 |
| VASH2 | 8.763 | 2.383 | 5.761 | 4.245 |
| RAG1 | 46.66 | 0.231 | 0.251 | 0.265 |
| S1PR1 | 5.222 | 1.058 | 0.737 | 0.461 |
| CD27-AS1 | 3.088 | 14.958 | 5.11 | 4.613 |
| SLIT1 | 17.333 | 2.481 | 2.6 | 2.815 |
| C3 | 0.006 | 3.907 | 0.489 | 1.329 |
| DPY19L2P2 | 6.664 | 2.117 | 2.937 | 2.643 |
| CCL4L1 | 0 | 13.27 | 0.087 | 0.328 |
| BAI2 | 4.519 | 0.893 | 1.395 | 1.079 |
| AIM1 | 26.38 | 7.777 | 37.047 | 27.486 |
| NFKBIE | 4.506 | 15.504 | 11.399 | 11.793 |
| HSPA5 | 78.621 | 501.993 | 373.499 | 449.173 |
| NKD2 | 15.011 | 3.784 | 3.385 | 3.105 |
| SLCO3A1 | 5.528 | 1.725 | 3.481 | 3.095 |
| IER3 | 0.506 | 91.488 | 31.73 | 26.899 |
| CLEC2B | 5.555 | 54.539 | 60.911 | 54.119 |
| NINJ1 | 10.319 | 76.622 | 11.769 | 54.452 |
| EIF2AK3 | 5.111 | 15.035 | 9.332 | 9.816 |
| ZC3H12A | 3.335 | 23.815 | 13.36 | 17.547 |
| MIR4435-1HG | 4.149 | 31.674 | 17.138 | 16.303 |
| CCL3L1 | 0 | 6.448 | 0 | 0.079 |
| ABCB10 | 51.637 | 9.932 | 10.164 | 9.751 |
| NT5M | 7.67 | 2.289 | 1.582 | 1.643 |
| MCM5 | 104.109 | 31.862 | 37.057 | 33.895 |
| DTX3L | 8.459 | 36.101 | 48.022 | 48.261 |
| DNAJB11 | 40.794 | 111.886 | 107.644 | 109.967 |
| LINC00977 | 10.816 | 0.925 | 0.741 | 0.266 |
| APOBEC3D | 2.393 | 13.069 | 10.187 | 8.981 |
| RAP1GAP2 | 3.998 | 0.092 | 0.368 | 0.285 |
| PPM1H | 11.904 | 2.973 | 4.492 | 4.178 |
| TNFSF9 | 1.393 | 7.107 | 2.568 | 2.651 |
| PEAK1 | 3.018 | 12.974 | 5.442 | 9.633 |
| BATF3 | 18.188 | 49.098 | 36.514 | 45.085 |
| POU3F2 | 3.711 | 0.566 | 1.044 | 0.696 |
| CST7 | 3.315 | 14.15 | 2.338 | 3.175 |
| MIR221 | 2.007 | 21.479 | 10.429 | 10.092 |
| CASC15 | 5.894 | 1.102 | 4.027 | 2.572 |
| CDT1 | 39.057 | 12.4 | 11.013 | 11.831 |
| FOXO1 | 1.167 | 8.247 | 1.77 | 3.736 |
| IL22 | 0 | 1.6 | 3.116 | 3.136 |
| BTG1 | 4.391 | 33.793 | 22.099 | 23.454 |
| ATG14 | 5.727 | 30.239 | 7.863 | 11.252 |
| NR4A2 | 0 | 10.173 | 0.04 | 0.356 |
| NAMPT | 8.894 | 108.843 | 34.628 | 54.272 |
| FOS | 0.145 | 3.108 | 7.329 | 4.53 |
| C10orf54 | 0.84 | 7.506 | 0.382 | 0.487 |
| YBX2 | 3.976 | 1.141 | 1.555 | 1.594 |
| LIME1 | 42.247 | 6.433 | 15.749 | 11.879 |
| BAIAP2-AS1 | 3.939 | 0.977 | 2.72 | 2.144 |
| CBX4 | 24.877 | 7.596 | 12.478 | 11.578 |
| IL23A | 2.262 | 20.398 | 12.207 | 17.874 |
| EPHA3 | 7.734 | 2.853 | 2.019 | 2.536 |
| MYO1D | 4.114 | 11.118 | 4.59 | 5.995 |
| JUN | 1.525 | 5.925 | 3.835 | 3.58 |
| APOBEC3A_B | 0.029 | 11.374 | 10.242 | 14.665 |
| STARD4 | 8.464 | 23.37 | 20.118 | 16.105 |
| NLRC5 | 2.969 | 26.889 | 38.784 | 37.7 |
| ETV5 | 0.596 | 9.791 | 15.576 | 13.264 |
| KBTBD11 | 6.611 | 2.011 | 2.434 | 2.772 |
| CD27 | 2.525 | 27.462 | 3.098 | 3.4 |
| KSR1 | 1.826 | 8.146 | 4.79 | 5.998 |
| KLF2 | 5.551 | 0.296 | 1.971 | 1.375 |

TABLE 3

| | | NC_P + | FK506_P + | CABIN1_P + |
|---|---|---|---|---|
| | NC/ | I/ | I/ | I/ |
| NAME | FPKM | FPKM | FPKM | FPKM |
| PDGFA | 1.497 | 26.809 | 3.25 | 8.169 |
| ZNF827 | 0.975 | 3.401 | 1.521 | 1.372 |
| MIR6821 | 17.553 | 62.894 | 13.626 | 25.116 |
| ADORA2A | 2.492 | 11.633 | 0.998 | 1.475 |
| GTDC1 | 3.595 | 10.378 | 4.629 | 5.144 |
| TBC1D4 | 20.739 | 73.749 | 12.549 | 13.191 |
| CSF1 | 0.848 | 13.43 | 1.706 | 2.594 |
| PTPN6 | 22.163 | 77.092 | 20.906 | 20.806 |
| IL2 | 0 | 397.807 | 0.287 | 12.964 |
| CCL3 | 0 | 52.239 | 0 | 0.502 |
| FOSL2 | 1.294 | 7.508 | 2.204 | 2.362 |
| CXCL8 | 0.037 | 26.755 | 0.937 | 3.327 |
| XCL1 | 0 | 49.113 | 0 | 1.144 |
| PAM | 1.154 | 5.577 | 1.483 | 1.934 |
| PPIF | 58.328 | 188.301 | 50.291 | 106.533 |
| SORBS1 | 0.196 | 5.08 | 0.4 | 0.511 |
| IL21R | 0.1 | 34.712 | 2.244 | 5.255 |
| LTBP4 | 7.304 | 33.466 | 8.427 | 9.568 |
| IQCJ-SCHIP1 | 1.402 | 4.253 | 1.657 | 1.234 |
| NFATC1 | 4.511 | 15.287 | 3.793 | 3.916 |
| PLS3 | 0.019 | 4.717 | 0.052 | 0.37 |
| VAMP1 | 9.566 | 50.97 | 14.793 | 14.752 |
| SLC35E4 | 0.887 | 9.069 | 0.952 | 1.517 |
| TNFSF14 | 0.045 | 69.958 | 1.023 | 4.255 |
| TIAM2 | 0.255 | 4.095 | 0.27 | 0.301 |
| FXYD5 | 25.643 | 101.352 | 41.346 | 42.856 |
| CD200 | 0 | 3.759 | 0 | 0.094 |
| POU2AF1 | 1.84 | 36.298 | 2.274 | 2.971 |
| NTRK1 | 0.167 | 7.941 | 0.836 | 1.657 |
| SCHIP1 | 1.25 | 3.933 | 1.535 | 1.333 |
| DSTYK | 2.356 | 7.198 | 3.501 | 3.595 |
| JAK2 | 2.788 | 26.566 | 5.248 | 8.45 |
| FYN | 14.671 | 58.084 | 16.523 | 15.512 |
| TAGAP | 3.351 | 128.498 | 4.352 | 8.731 |
| GFOD1 | 2.283 | 12.861 | 4.187 | 6.312 |
| LIF | 0.063 | 3.697 | 0.143 | 0.231 |
| COL27A1 | 4.149 | 17.216 | 4.463 | 5.729 |
| PHACTR2 | 1.659 | 9.467 | 2.927 | 2.791 |
| SPRY1 | 2.079 | 12.391 | 4.074 | 4.901 |
| BCL2L1 | 31.375 | 113.956 | 39.402 | 41.852 |
| XCL2 | 0.056 | 70.747 | 0 | 2.074 |
| KCNK1 | 10.828 | 36.893 | 10.967 | 13.139 |
| CREB5 | 0.031 | 4.533 | 0.577 | 1.966 |
| SLAMF1 | 0.042 | 5.663 | 0.217 | 0.275 |
| MIR6775 | 12.409 | 50.527 | 19.643 | 21.57 |
| ARHGEF3 | 6.146 | 31.337 | 8.637 | 8.674 |
| CSF2 | 0 | 23.497 | 0 | 0.314 |
| AGK | 14.395 | 39.056 | 14.029 | 14.306 |
| CD97 | 10.97 | 49.481 | 19.394 | 21.496 |
| IRF4 | 0.034 | 8.439 | 0.423 | 2.092 |
| NPDC1 | 3.008 | 8.759 | 4.328 | 5.64 |
| CCL20 | 0 | 59.393 | 0 | 1.279 |
| CABP1 | 1.702 | 5.834 | 1.201 | 1.689 |
| ZNF704 | 0.107 | 4.985 | 0.014 | 0.183 |
| CD70 | 1.101 | 61.146 | 3.005 | 7.606 |
| TDRD9 | 1.428 | 25.902 | 1.245 | 1.888 |
| IL21R-AS1 | 0.048 | 23.616 | 1.927 | 3.991 |
| ATP6V1B2 | 33.172 | 129.395 | 50.672 | 60.661 |
| CCL3L3 | 0 | 6.524 | 0 | 0.079 |
| TM4SF1 | 0 | 3.268 | 0.439 | 0.907 |
| RGS16 | 0.077 | 29.102 | 1.172 | 5.595 |
| IL3 | 0 | 229.054 | 0.062 | 6.298 |
| LAX1 | 8.344 | 24 | 8.052 | 7.578 |
| SERPINB2 | 0.28 | 85.882 | 2.905 | 14.103 |
| CCDC64 | 5.477 | 33.545 | 10.044 | 11.039 |
| LRRC8B | 2.89 | 28.699 | 3.555 | 4.298 |
| STAT5A | 1.417 | 9.105 | 3.347 | 3.055 |
| SHC4 | 0.069 | 4.391 | 0.663 | 0.686 |
| ASAP1 | 4.678 | 18.37 | 7.765 | 9.052 |
| PTPN22 | 8.928 | 45.38 | 6.37 | 5.119 |
| TNFRSF4 | 2.597 | 31.683 | 2.933 | 11.916 |
| NDFIP1 | 19.638 | 54.615 | 22.485 | 24.432 |
| LAMP3 | 8.064 | 31.61 | 6.412 | 9.828 |
| LOC729603 | 1.468 | 5.071 | 2.179 | 3.329 |
| PKIA | 6.444 | 49.019 | 13.962 | 18.138 |
| CD55 | 3.587 | 39.451 | 6.015 | 12.082 |
| PHEX | 0.188 | 17.377 | 0.213 | 0.786 |
| GRK5 | 5.451 | 21.464 | 8.481 | 8.975 |
| FMNL2 | 2.395 | 6.813 | 2.726 | 3.019 |
| CCL4L2 | 0 | 12.758 | 0.087 | 0.328 |
| CRIM1 | 0.268 | 3.029 | 0.305 | 0.333 |
| ISM1 | 0.798 | 56.893 | 0.726 | 3.737 |
| FBXO34 | 11.735 | 48.015 | 14.612 | 18.386 |
| MAP2K3 | 21.676 | 144.057 | 41.815 | 43.237 |
| TBC1D20 | 11.844 | 35.808 | 17.7 | 18.427 |
| CCL1 | 0 | 9.86 | 0 | 0.415 |
| PIM3 | 37.434 | 180.382 | 42.827 | 72.895 |
| RDH10 | 4.125 | 19.889 | 5.856 | 5.524 |
| CRTAM | 0.104 | 100.175 | 3.325 | 9.51 |
| PELI1 | 5.755 | 16.085 | 8.175 | 8.465 |
| CCND2 | 0.321 | 6.436 | 0.558 | 0.99 |
| CXCR3 | 0.604 | 14.322 | 0.503 | 1.803 |
| IRF8 | 1.168 | 14.813 | 1.19 | 1.914 |
| REL | 2.654 | 20.219 | 4.749 | 5.699 |
| CCL4 | 0 | 247.287 | 0 | 4.062 |
| KCNK5 | 3.84 | 14.865 | 3.906 | 4.904 |
| PLK3 | 4.432 | 14.807 | 5.257 | 7.036 |
| TNS3 | 0.008 | 13.196 | 0.678 | 1.418 |
| CD27-AS1 | 3.088 | 14.958 | 5.11 | 4.613 |
| C3 | 0.006 | 3.907 | 0.489 | 1.329 |
| CCL4L1 | 0 | 13.27 | 0.087 | 0.328 |
| CCL3L1 | 0 | 6.448 | 0 | 0.079 |
| TNFSF9 | 1.393 | 7.107 | 2.568 | 2.651 |
| CST7 | 3.315 | 14.15 | 2.338 | 3.175 |
| FOXO1 | 1.167 | 8.247 | 1.77 | 3.736 |
| ATG14 | 5.727 | 30.239 | 7.863 | 11.252 |
| NR4A2 | 0 | 10.173 | 0.04 | 0.356 |
| C10orf54 | 0.84 | 7.506 | 0.382 | 0.487 |
| MYO1D | 4.114 | 11.118 | 4.59 | 5.995 |
| CD27 | 2.525 | 27.462 | 3.098 | 3.4 |

2.5 CABIN1 Peptide that does not Inhibit Calcineurin Phosphatase Activity

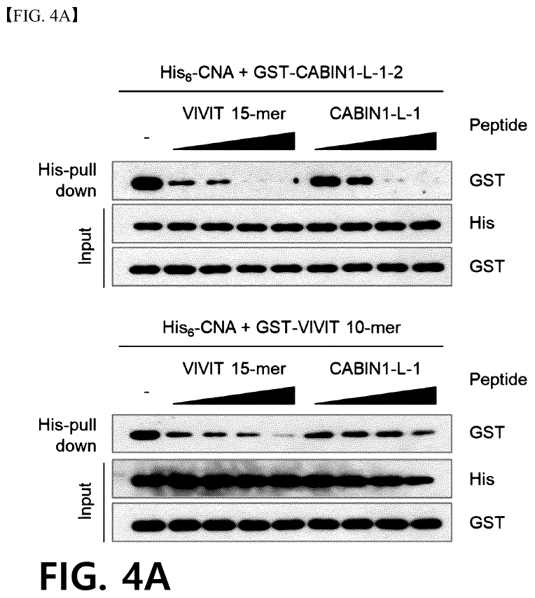

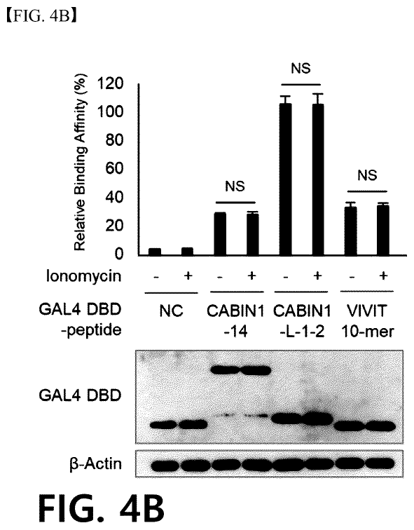

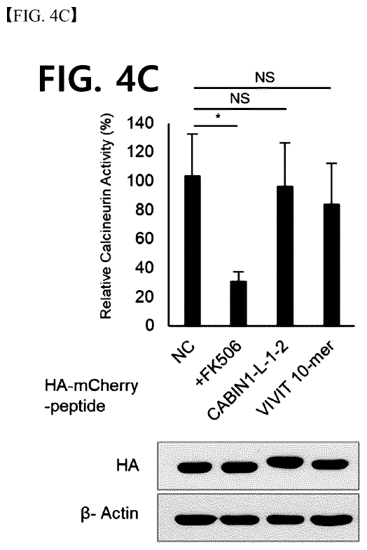

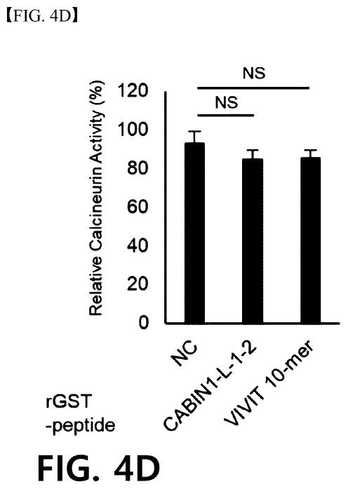

VIVIT peptide is known to interfere with CNA-NFAT interaction by binding to regions other than the CNA catalytic site (Takeuchi, et al., K., Structure 15, 587-597 (2007)). To determine whether CABIN1 and VIVIT peptides have the same mechanism of action, a peptide competition assay was performed. CABIN1 peptide dose-dependently interfered with the interaction between GST-tagged VIVIT peptide and His6-tagged CNA and vice versa (FIG. 4A). In addition, ionomycin treatment did not affect the interaction between CNA and CABIN1 or VIVIT peptides (FIG. 4B). Considering that the autoinhibitory domain of CNA blocks the active site under normal conditions and that the active site is exposed through calcium-induced structural changes during ionomycin treatment, this result confirms that CABIN1 peptide, VIVIT peptide, and fused tag (GAL4 DBD) do not interfere with the active site of CNA. Thereafter, calcineurin phosphatase activity was measured in Jurkat T cells expressing each peptide or treated with FK506 (FIG. 4C). FK506, which interferes with the active site of CNA, reduced the phosphatase activity of CNA, but CABIN1 and VIVIT peptides did not. In addition, these peptides did not reduce the phosphatase activity of CNA in in vitro experiments (FIG. 4D).

2.6 C-Terminus PPTP Sequence that is Important for Interaction with Calcineurin

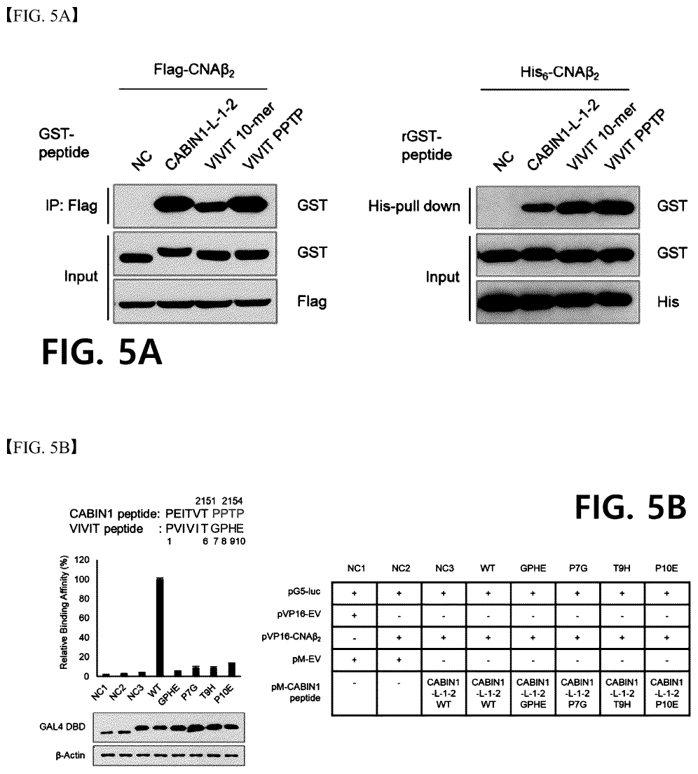

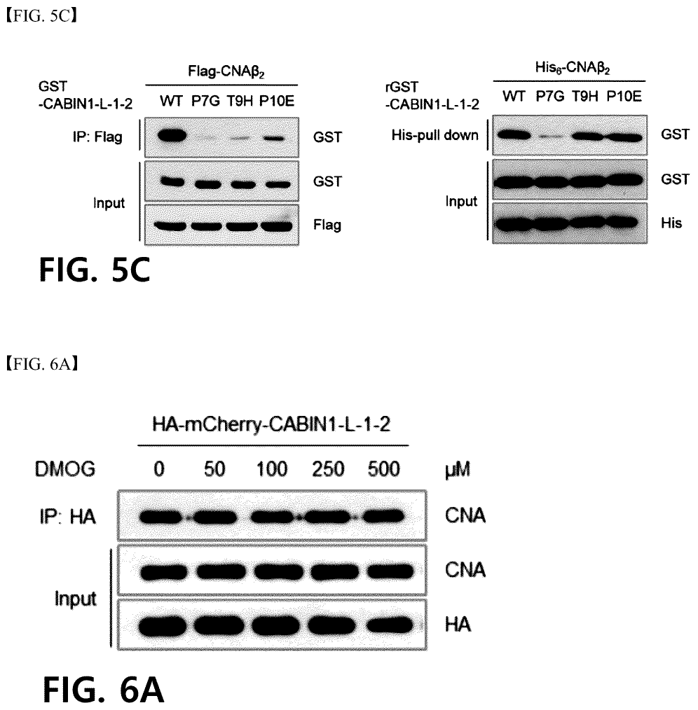

To understand how the PPTP sequence among the peptide sequences of the present invention contributes to the binding affinity for CNA, the binding affinity of each GST-tagged peptide for CNA at the cell level and in vitro was compared (FIG. 5A). The PPTP sequence enhanced CNA binding affinity only at the cellular level, not in vitro, suggesting that this effect was due to post-translational modification (PTM) of the “PPTP” residues. Thereafter, one or all of the PPTP residues were substituted with amino acids corresponding to each residue of the GPHE sequence of the VIVIT peptide. A mammalian two-hybrid assay was performed using peptides of these sequences and CNA to determine which modifications influence affinity (FIG. 5B). As a result, all substitutions dramatically interfered with the interaction with CNA, meaning that all residues in the “PPTP” sequence are important for the interaction with CNA. This result suggests that the “PPTP” sequence may be a substrate motif of an upstream enzyme capable of modifying residues. In vitro, peptides substituted with threonine 9 and proline 10 residues showed similar binding affinity with wild-type CABIN1 peptides for CNA, but in cell experiments, all substitutions interfered with their interactions with calcineurin (FIG. 5C). This suggests that threonine 9 and proline 10 of the CABIN1 peptide may be targets of PTM.

2.7 Phosphorylation of CABIN1 T2154 that is Important for Interaction with Calcineurin

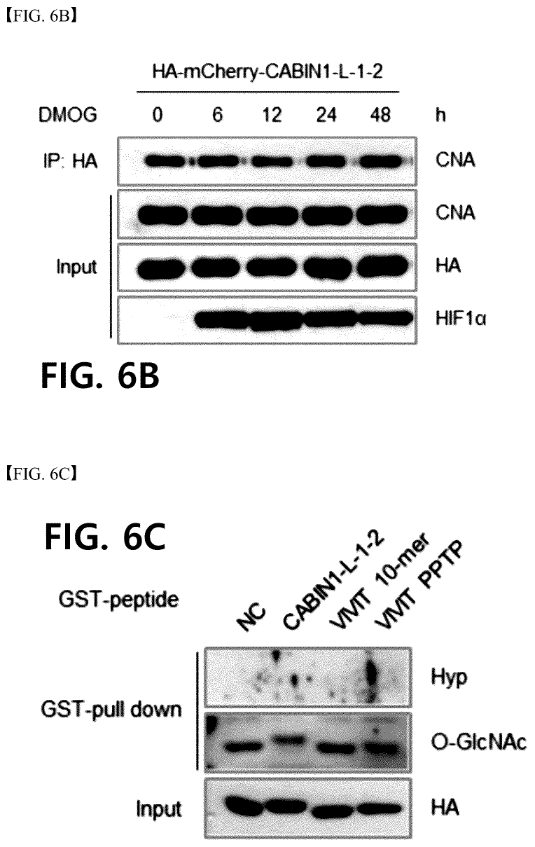

Phosphorylation and O-linked glycosylation of threonine residues and hydroxylation of proline residues of PPTP-containing peptides were investigated. First, it was investigated whether the proline residue of “PPTP” was hydroxylated. Jurkat T cells expressing mCherry-tagged CABIN1 peptide were treated with dimethyloxalylglycine (DMOG) which is a prolyl hydroxylase inhibitor (FIGS. 6A, 6B). DMOG stabilized HIF-1α (hypoxia-inducible factor-1 alpha) but had no effect on the interaction between CABIN1 peptide and CNA. In addition, it did not detect hydroxyproline in GST-tagged CABIN1 peptide or VIVIT peptide (FIG. 6C). Subsequently, O-linked glycosylation was checked at threonine residues in PPTP. O-linked glycosylation of GST-tagged peptide containing PPTP (CABIN1-L-1-2 and VIVIT PPTP) was not higher than that of negative control or VIVIT peptide, indicating that no O-linked glycosylation occurred in the PPTP sequence (FIG. 6C).

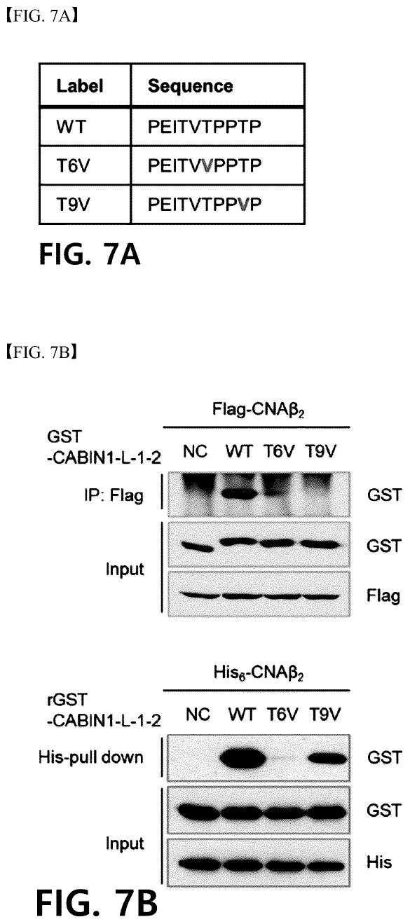

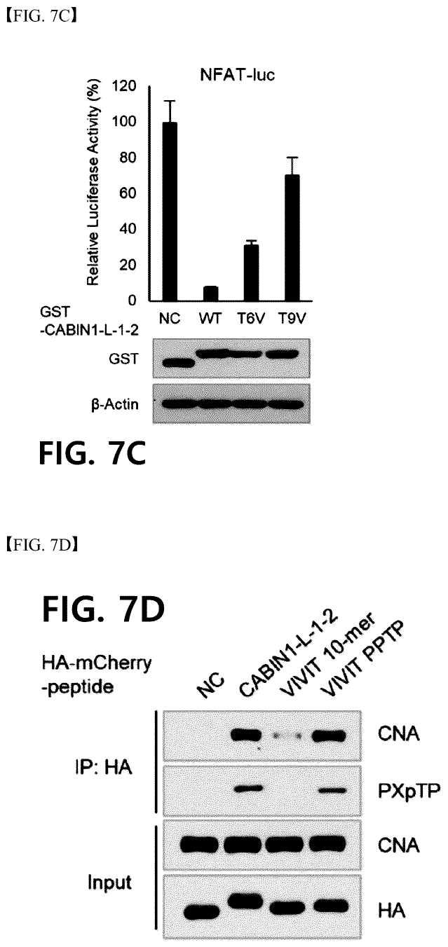

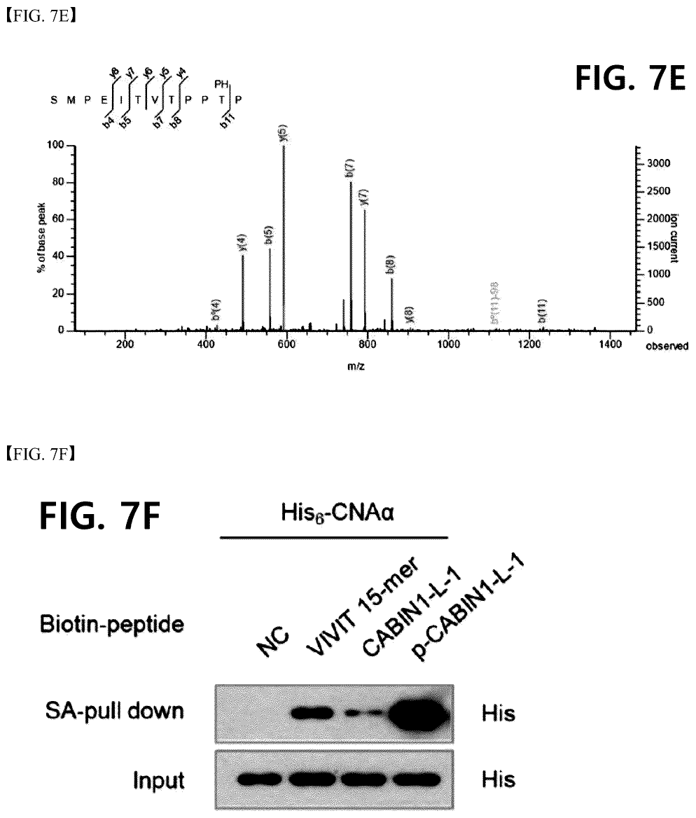

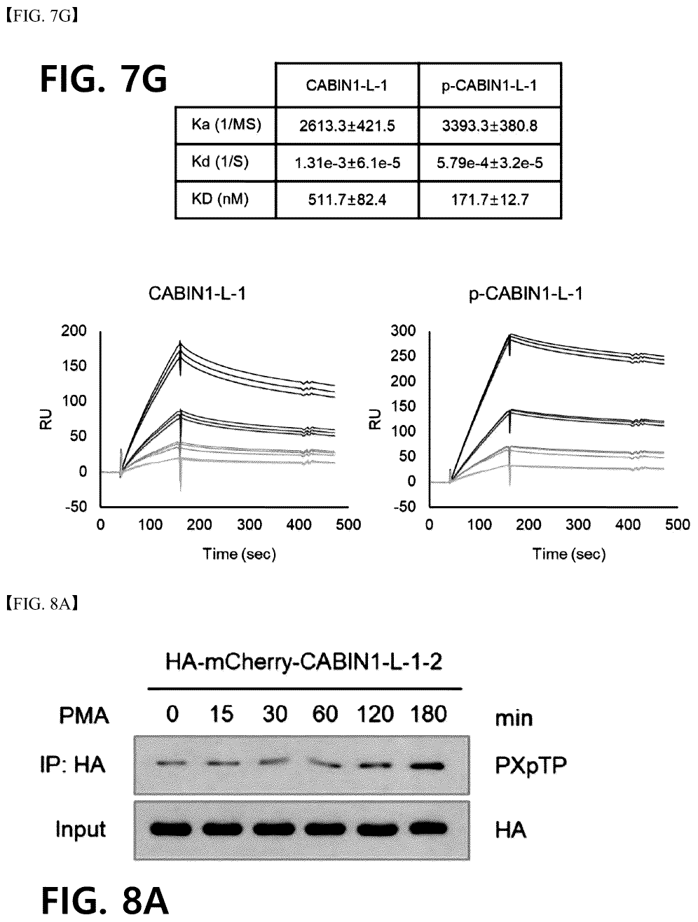

To identify post-transcriptionally modified residues in the CABIN1 peptide sequence, each threonine was replaced with valine containing a methyl group instead of the hydroxyl group of threonine (FIG. 7A). Replacing threonine 6 (threonine 2151 of full-length CABIN1) with valine (T6V) resulted in the loss of interaction between the CABIN1 peptide and CNA in cells and in vitro. In contrast, replacing threonine 9 (threonine 2154 of full-length CABIN1) with valine (T9V) resulted in the loss of interaction in cell experiments but not in vitro (FIG. 7B). Moreover, the T9V substitution restored the transcriptional activity of NFAT inhibited by the wild-type CABIN1 peptide more efficiently than the T6V substitution (FIG. 7C). These results suggest that PTM occurs on threonine 9, which increases the inhibitory effect on the CN-NFAT pathway.

Threonine 9 phosphorylation was detected by Western blotting using antibodies against phospho-MAPK substrates (PXpTP) in Jurkat T cells expressing mCherry-tagged peptide (FIG. 7D). To directly detect phosphorylated CABIN1 peptides in cells, those peptides were immunoprecipitated with anti-HA antibodies in Jurkat T cells expressing HA-mCherry-tagged CABIN1 peptide. The purified protein was analyzed by LC-MS/MS to observe threonine 9 phosphorylation (FIG. 7E). Subsequently, biotin-labeled VIVIT peptides, CABIN1 peptides, and phosphorylated CABIN1 peptides were synthesized. Streptavidin pull-down assay of these peptides showed that CABIN1 phosphorylation dramatically increased the affinity for CNA (FIG. 7F). Finally, the binding ability was quantitatively analyzed through Surface Plasma Resonance (SPR). It was observed that phosphorylation of the ninth threonine of the CABIN1 peptide increased the binding affinity approximately three-fold (FIG. 7G). Collectively, these results demonstrate that the CABIN1 peptide is a more efficient in vivo inhibitor than the VIVIT peptide because phosphorylation of threonine in PPTP enhances binding affinity for CNA.

2.8 Threonine 9 Phosphorylation of CABIN1 Peptide by p38MAPK

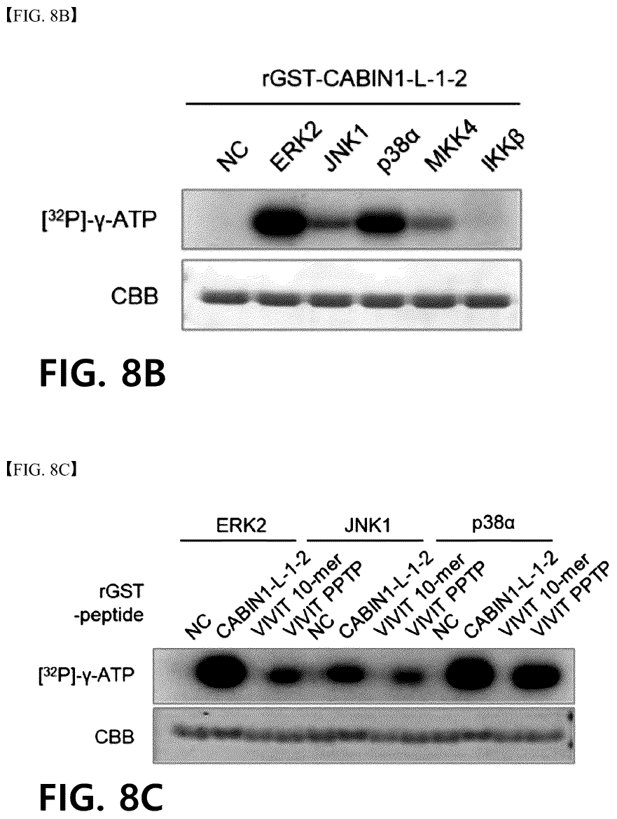

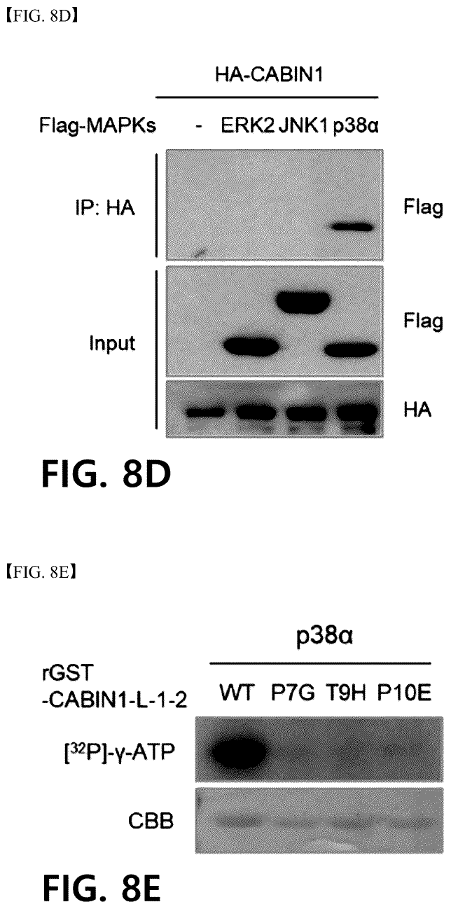

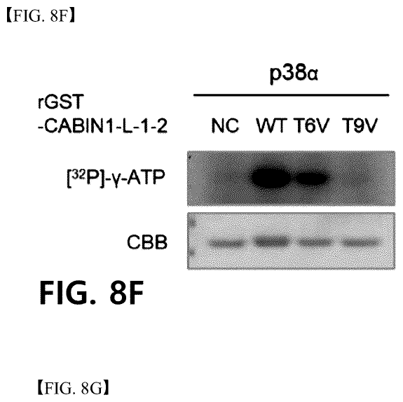

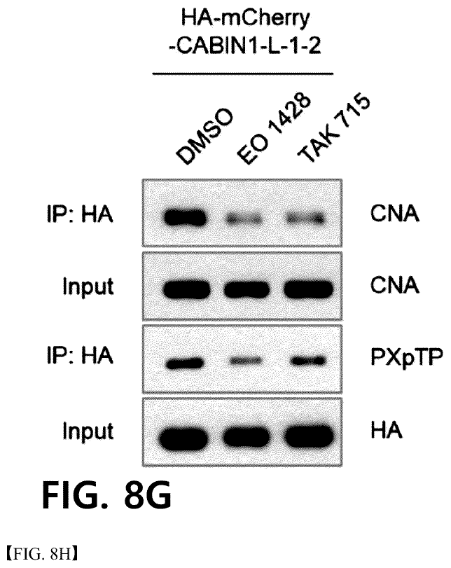

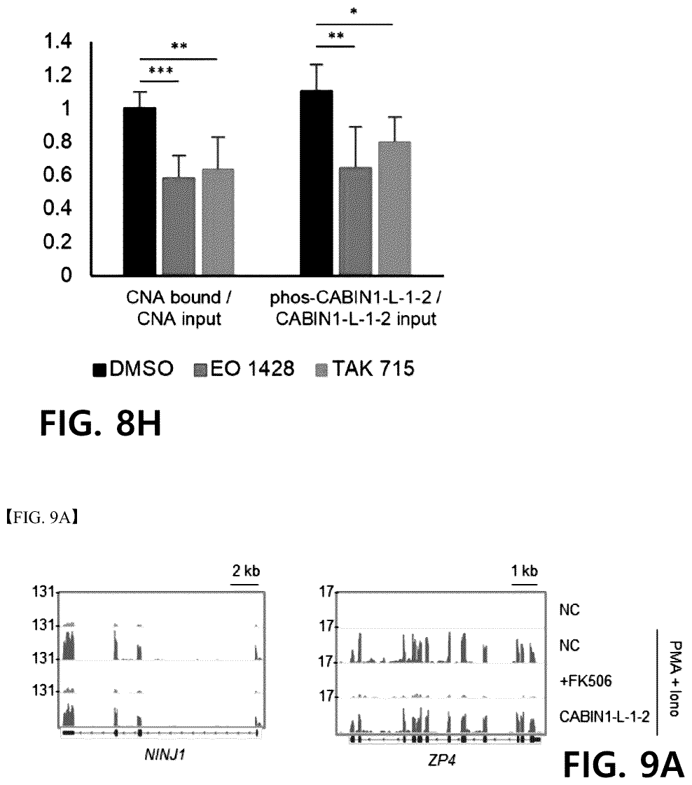

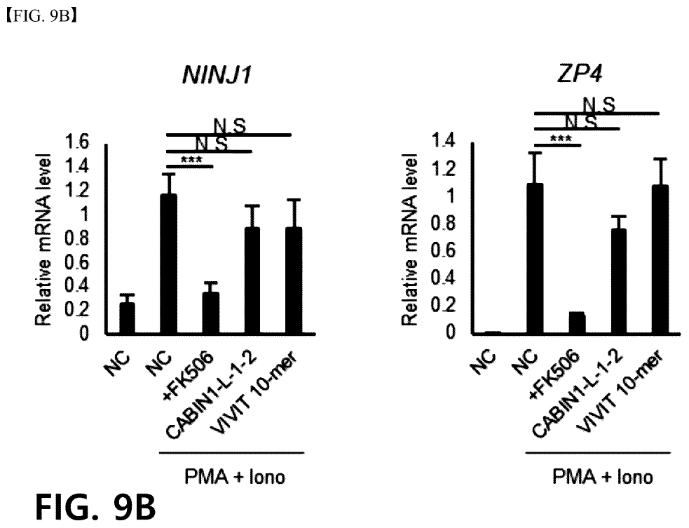

PMA, an activator of PKC, induces hyperphosphorylation of CABIN1, but PKC does not directly phosphorylate CABIN1. Therefore, we investigated whether PMA causes threonine phosphorylation of CABIN1 peptide, especially PPTP, and which kinase causes phosphorylation. After PMA treatment, CABIN1 peptide phosphorylation was increased 2 hours after treatment (FIG. 8A). To identify kinase capable of directly phosphorylating CABIN1 peptide, an in vitro phosphorylation assay was performed with kinase candidates activated by PKC. It was found that the MAPK family (EKR, JNK, and p38) and MKK4 directly phosphorylate CABIN1 peptide (FIG. 8B). Considering that PPTP is a perfectly suitable substrate motif for MAPK family, it was focused on them. ERK2, JNK1, and p38a phosphorylate purified recombinant GST-tagged peptide containing PPTP sequence (CABIN1-L-1-2 and VIVIT PPTP), but not GST-tagged VIVIT peptide (FIG. 8C). An immunoprecipitation assay was performed to investigate kinase interacting with CABIN1. This assay showed that only p38a was bound to CABIN1 at the cellular level (FIG. 8D). In addition, p38a did not phosphorylate the substituted PPTP sequence in vitro (FIG. 8E, FIG. 8F). Thereafter, it was confirmed that selective p38 inhibitors such as EO 1428 or TAK 715 significantly reduced phosphorylation of the CABIN1 peptide, thereby reducing binding affinity for CNA (FIG. 8G, FIG. 8H). These results suggest that p38a is in fact an upstream kinase of the CABIN1 peptide.

As above, specific parts of the present invention have been described in detail, and it will be clear to those skilled in the art that these specific techniques are merely preferred embodiments and do not limit the scope of the present invention. Accordingly, the substantial scope of the present invention will be defined by the appended claims and their equivalents.

What is claimed is:

1. A peptide for inhibiting calcineurin, comprising 8 to 10 consecutive amino acids of the following General Formula 1:
P-X₁-I-X₂-X₃-T-P-P-T-P-T, [General Formula 1]
wherein in General formula 1,
X₁ is valine (V) or glutamic acid (E),
X₂ is valine (V) or threonine (T), and
X₃ is valine (V) or isoleucine (I).

2. The peptide for inhibiting calcineurin of claim 1, wherein the consecutive amino acids comprise the amino acids of SEQ ID NO: 1.

3. The peptide for inhibiting calcineurin of claim 1, wherein the consecutive amino acids comprise the amino acids of SEQ ID NO: 2 or SEQ ID NO: 3.

4. The peptide for inhibiting calcineurin of claim 1, wherein the peptide further comprises at least one amino acid selected from the group consisting of alanine (A) glycine (G), proline (P) histidine (H), and phenylalanine (F) proline (P) at the N-terminus of the amino acid of General formula 1.

5. The peptide for inhibiting calcineurin of claim 1, wherein the peptide is one in which threonine (T) residue of the amino acid of General Formula 1 is phosphorylated.

6. The peptide for inhibiting calcineurin of claim 5, wherein the threonine (T) is threonine (T) of SEQ ID NO: 1.

7. The peptide for inhibiting calcineurin of claim 5, wherein the phosphorylation is induced by p38MAPK.

8. The peptide for inhibiting calcineurin of claim 5, wherein the phosphorylation occurs within T cells.

9. The peptide for inhibiting calcineurin of claim 1, wherein the peptide inhibits the proliferation of T cells.

10. The peptide for inhibiting calcineurin of claim 1, wherein the peptide is derived from CABIN1 peptide.

11. The peptide for inhibiting calcineurin of claim 1, wherein the peptide inhibits CN-NFAT pathway.

12. A pharmaceutical composition for immunosuppression comprising the peptide of claim 1 as an active ingredient.

13. A pharmaceutical composition for preventing or treating immune diseases or inflammatory diseases, comprising the peptide of claim 1 as an active ingredient.

14. The pharmaceutical composition of claim 13, wherein the immune disease is at least one selected from the group consisting of: rheumatoid arthritis, Bechcet's disease, multiple myositis or skin myositis, autoimmune hemocytopenia, autoimmune myocarditis, atopic dermatitis, asthma, primary cirrhosis, dermatomyositis, Goodpasture's syndrome, autoimmune meningitis, Sjogren's syndrome, lupus, Addison's disease, alopecia areata, ankylosing spondylitis, autoimmune hepatitis, autoimmune parotitis, Crohn's disease, insulin-dependent diabetes, dystrophic epidermolysis bullosa, epididymitis, glomerulonephritis, Graves' disease, Guillain-Barré syndrome, Hashimoto's disease, hemolytic anemia, multiple sclerosis, myasthenia gravis, pemphigus vulgaris, psoriasis, rheumatic fever, sarcoidosis, scleroderma, spondyloarthropathy, thyroiditis, vasculitis, vitiligo, myxedema, pernicious anemia, mitochondria-related syndrome, and ulcerative colitis.

15. The pharmaceutical composition of claim 13, wherein the inflammatory disease is at least one selected from the group consisting of: edema, allergy, periodontitis, rhinitis, otitis media, sore throat, tonsillitis, pneumonia, gastric ulcer, gastritis, colitis, hemorrhoids, gout, ankylosing spondylitis, arthritis, osteoarthritis, periarteritis, tendinitis, tenosynovitis, myositis, hepatitis, cystitis and nephritis.
